(19) United States
(12) Patent Application Publication
(10) Pub. No.: US 2009/0093069 A1
(43) Pub. Date: Apr. 9, 2009

(54) Topiramate Immunoassays

(75) Inventors: Johnny Valdez, Fremont, CA (US); Byung Sook Moon, Palo Alto, CA (US); Jacqueline Nguyen, Milpitas, CA (US); Alejandro Orozco, Gilroy, CA (US)

Correspondence Address: MORGAN, LEWIS & BOCKIUS LLP (SF), One Market, Spear Street Tower, Suite 2800, San Francisco, CA, 94105, US

(73) Assignee: ARK Diagnostics, Inc., Sunnyvale, CA (US)

(21) Appl. No.: 12/135,145

(22) Filed: Jun. 7, 2008

Related U.S. Application Data
(60) Provisional application No. 60/978,706, filed on Oct. 9, 2007.

(30) Foreign Application Priority Data
Jun. 6, 2008 (US) PCT/US08/66224

(51) Int. Cl. G01N 33/566; C07K 16/00; C07D 493/14

(52) U.S. Cl. 436/518; 530/389.1; 549/387

(57) ABSTRACT

Diacetonefructose derivatives have substituents at the hydroxyl-position. Diacetonefructose derivatives may include immunogenic moieties to prepare anti-diacetonefructose derivative antibodies, or antigenic moieties for immunodiagnostic assays. Also, the diacetonefructose derivatives can include signal generating moieties for detecting the presence or amount of the diacetonefructose derivative in a sample. Additionally, the diacetonefructose derivatives can be used in immunodiagnostic assays to compete with topiramate for binding with anti-diacetonefructose derivative antibodies. Also, methods, compositions and kits are disclosed directed at diacetonefructose derivatives, immunogens, signal generating moieties and immunoassays for topiramate.

### CROSS-REFERENCE TO RELATED APPLICATIONS

This application claims the benefit of U.S. application Ser. No. 11/760,409, filed Jun. 8, 2007, for which a petition to convert non-provisional application to provisional application has been filed; and U.S. Provisional Application No. 60/978,706, filed Oct. 9, 2007, each of which is incorporated by reference in its entirety for all purposes.

### FIELD OF THE INVENTION

The present invention relates to diacetonefructose derivatives for use as immunodiagnostic reagents in detecting or quantifying topiramate in biological fluids or tissue. More particularly, the present invention relates to diacetonefructose derivatives, immunogens and antigens prepared from diacetonefructose derivatives, antibodies prepared from diacetonefructose derivatives-based immunogens, and methods of making and using the same in detecting or quantifying topiramate in biological fluids or tissue.

### BACKGROUND OF THE INVENTION

Topiramate is the generic name for 2,3:4,5-bis-O-(1-methylethylidene)-β-D-fructopyranose sulfamate:

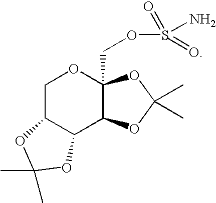

Topiramate is an anti-epileptic drug (“AED”), and is chemically unrelated to many existing AEDs. Topiramate, which is the active ingredient in TOPAMAX®, was approved by the FDA in 1996 for use as adjunctive therapy in the treatment of adults with partial seizures with or without secondary generalization, and may also be useful for Lennox-Gastaut syndrome and infantile spasms. It is also indicated for prophylaxis of migraine headache.

Following oral ingestion, the absorption of topiramate is rapid (Tₘₐₓ, 2-4 h), with a bioavailability of 81-95% (Easterling et al., 1988). The Vd of topiramate is 0.6-1 L/kg. Topiramate is only 15% bound to serum proteins, but it does have a high affinity/low capacity binding site on erythrocytes (Doose & Streeter, 2002). There is a linear relationship between topiramate dose and serum concentration. Approximately 50% of the dose is metabolized. Topiramate oxidized metabolites, including 9-hydroxytopiramate, 10-hydroxytopiramate, 2,3-diol topiramate, and 4,5-diol topiramate, can be found in plasma and urine (See FIG. 1, and Malka et al., 2005). Topiramate has a serum half-life of 20-30 h, but in patients co-prescribed enzyme-inducing AEDs, the hepatic metabolism of topiramate becomes more important, causing a shortening of the half-life to about 12 h, an increase in clearance, and a corresponding decrease in serum topiramate concentrations by approximately 50% (Sachdeo et al., 1996; Britzi et al., 2005). Topiramate is eliminated at a faster rate in children, the magnitude of the increase in clearance compared with adults ranges in different studies from 25% to 170% (Rosenfeld et al., 1999; Perucca, 2006).

Enzyme-inducing AEDs reduce serum topiramate concentrations by approximately 50% (Britzi et al., 2005, Mimrod et al., 2005), while valproic acid lowers topiramate concentrations by 10-15% (Rosenfeld et al., 1997). Topiramate clearance can be decreased by propranolol, amitriptyline, lithium, and sumatriptan resulting in slightly increased serum topiramate concentrations (Patsalos, 2005).

Reife et al. (1995) measured serum concentrations in three double-blind placebo controlled add-on trials, and the results suggested that seizure control was improved at serum topiramate concentrations in the narrow range of 3.5-5 mg/L (10-15 μmol/L). Another study comparing three serum concentration ranges (<1.7 mg/L; 1.7-10 mg/L; >10 mg/L), indicated that median seizure-free duration was significantly correlated with topiramate concentrations (Twyman et al., 1999). Penovich et al. (1997) showed a trend for higher serum topiramate concentrations (>10 mg/L) in patients with improvement compared with those without seizure reduction. Lhatoo et al. (2000), on the other hand, reported mean topiramate serum concentrations of 7 mg/L in responders and 9 mg/L in seizure-free patients, compared with 6 mg/L in patients in whom topiramate was stopped because of adverse effects, with a wide variation in the relationship between serum drug concentration and therapeutic or toxic effects. In another study, a wide range of doses and serum concentrations produced a favorable response in 70% of 170 patients with drug refractory (mostly partial) epilepsy, with 23% becoming seizure-free (Stephen et al., 2000). Serum topiramate concentrations in this group ranged between 2.4 and 18 mg/L. More recently, the results of a tripleblind, concentration-controlled, parallel-group trial which randomized 65 patients to one of three prespecified serum concentrations (low=2 mg/L, medium=10 mg/L and high=19 mg/L) were reported (Christensen et al., 2003). Patients assigned to the medium serum concentration had the best outcome with regard to seizure reduction, and those in the medium and high groups experienced more adverse events than patients in the low group. The authors concluded that an optimal response is most likely to be achieved at serum concentrations of 2-10 mg/L. May et al. (2002) reported topiramate concentrations in the range of 2.4-8 mg/L in patients receiving topiramate doses of 125-400 mg in addition to other AEDs. Some patients receiving considerably higher doses (up to 2000 mg/day) had topiramate concentrations as high as 27 mg/L but no mention was made as to whether or not these concentrations were well tolerated. Overall, in most studies published to date, serum topiramate concentrations in the order of 5-20 mg/L have been reported in patients treated with therapeutic doses (Johannessen et al., 2003). Since in recent years there has been a trend toward using dosages lower than those tested in the initial studies, most patients will probably have concentrations in the low to mid portion of that range.

Situations in which AED therapeutic drug monitoring (“TDM”) are most likely to be of benefit have been described by Patsalos et al., Epilepsia; (published online Apr. 4, 2008). They include (1) when a person has attained the desired clinical outcome, to establish an individual therapeutic concentration which can be used at subsequent times to assess potential causes for a change in drug response; (2) as an aid in the diagnosis of clinical toxicity; (3) to assess compliance, particularly in patients with uncontrolled seizures or breakthrough seizures; (4) to guide dosage adjustment in situations associated with increased pharmacokinetic variability (e.g., children, the elderly, patients with associated diseases, drug formulation changes); (5) when a potentially important pharmacokinetic change is anticipated (e.g., in pregnancy, or when an interacting drug is added or removed); and (6) to guide dose adjustments for AEDs with dose-dependent pharmacokinetics.

Specifically, several characteristics of topiramate suggest there is a clinical need to individualize patient therapy by use of TDM. It has been suggested that there are large inter-individual variations in dose versus serum concentrations in patients. Also, pharmacokinetic variability plays a major role in the topiramate dosage requirements that are needed to achieve optimum serum concentrations.

It has been suggested that an appropriate range of optimal serum concentrations for topiramate would be 7 to 24 μmol/L in patients receiving a topiramate dose of 125 to 400 mg in addition to other AEDs. Some patients receiving considerably higher doses, which can be up to 2000 mg, had systemic topiramate concentrations as high as 80 μmol L. Effective TDM can be used to predict dosing regimens that can obtain appropriate topiramate concentrations within the therapeutic index. For conversion of units, topiramate concentration in μmol/L equals 2.95 times the topiramate concentration in mg/L.

Additionally, dose escalation add-on studies have been performed with topiramate with the intention of proceeding to monotherapy where possible. Accordingly, morning serum topiramate concentrations were taken and related to seizure control and associated side effects. Results indicated a clear improvement in seizure control with serum topiramate concentration in the range of 15 to 75 μmol/L, but a reduction in seizure control was seen at serum concentrations greater than 75 μmol/L. Also, there was a significant increase in side effects with serum concentrations greater then 60 μmol/L. Thus, a tentative target serum concentration range for topiramate of about 15 to 60 μmol/L has been suggested; however, most patients can have serum concentrations in the low to mid range with an appropriate dose regimen.

Many methods have been described for determining the systemic concentration of topiramate in a patient. See, Berry D J, et al. Ther Drug Monit; 22:460-4 (2000). Capillary gas chromatographic methods have described the determination of topiramate in serum using flame-ionizing detection and nitrogen-specific detection. See, Holland et al., J Chromatogr; 433:276-281 (1988), and Riffits et al., J Pharm Biomed Anal; 19:363-371 (1999), Tang et al., Ther Drug Monitoring; 22:195-201 (2000). Additionally, methods for using GLC or HPLC with MS have been shown to measure topiramate concentrations. See, Mozayani A, et al. J Anal Toxicol; 23:556-558 (1999), Chen S. et al., J Chromatogr; 761: 133-7 (2001), and Christensen et al., Ther Drug Monitoring; 24:658-664 (2002). However, such methods are impractical for commercial use due to, for example, long sample preparation time, long assay time, high cost, and labor-intensive procedures. Thus, a simple and fast analytical method for measuring topiramate serum (or plasma) levels is needed for effective TDM.

Immunoassay techniques have been developed to detect various drugs in biological samples and are well suited for such commercial analytical applications. Accordingly, immunoassays can be used to quickly determine the amount of a drug and/or drug metabolite in a patient's blood. Examples of immunoassays can include, but not limited to, homogeneous microparticle immunoassay (e.g., immunoturbidimetric), or quantitative microsphere systems (“QMS™”), fluorescence polarization immunoassay (“FPIA”), cloned enzyme donor immunoassay (“CEDIA”), chemiluminescent microparticle immunoassay (“CMIA”), enzyme multiplied immunoassay test (“EMIT”) and the like.

TDM immunoassays must be specific for the target drug. Patients with epilepsy may suffer from renal or hepatic diseases that interfere with their antiepileptic drug (AED) treatment. See, Lacerda G et al., Neurology; 67:S28-S33; 2006. Drugs excreted by the kidney, such as gabapentin, levetiracetam, and topiramate, have prolonged half-lives when patients with renal failure are not undergoing hemodialysis. The clinician needs to adjust dosages. Also, accumulation of drug metabolites in patients with renal dysfunction increases the potential for their crossreaction with therapeutic drug immunoassays (see Oellerich M et al., Clinical Chemistry; 47: 805-806; 2001) and may be a general and unappreciated occurrence. It is therefore important for drug immunoassay systems to have low cross-reactivity to non-active metabolites because additional cross-reactivity of drug metabolites in immunoassays can produce falsely increased drug concentrations.

A commercial reagent-based fluorescence polarization immunoassay (FPIA) technique is available for the measurement of topiramate in plasma or serum. See, U.S. Pat. No. 5,952,187, which is included herein by reference. Cross-reactivity studies were conducted to examine the cross-reactivity of the FPIA antiserum to structurally related compounds. 9-hydroxytopiramate demonstrated 6.9% to 13% cross-reactivity (product package insert Seradyn INNOFLUOR® TOPIRAMATE). Also, the FPIA immunoassay is limited to FPIA instrumentation such as the Abbott TDx® analyzer and by poor availability of previous topiramate analogs. The TDx systems are not high-volume throughput systems, so high-volume reference laboratories would prefer to use high throughput systems. It is desirable to have homogeneous methods that can be carried out quickly and simply, and permit, in particular, the automation and random access of sample analyses. For example, in high volume screening applications it can be desirable to have fully automated methods of analysis. As such, instruments are designed to detect changes in reaction rates and enzyme immunoassay reagents permit the complete automation and are applicable to many clinical chemistry analyzers found in reference and hospital clinical laboratories.

Another immunoassay commercially available is a homogeneous particle-enhanced turbidimetric immunoassay used for the analysis of topiramate in serum or plasma. See, U.S. Patent Application Publication 2008/0009018, which is incorporated herein by reference. The assay is based on competition between drug in the sample and drug coated onto a microparticle for antibody binding sites of the anti-topiramate antibody reagent. The topiramate coated microparticle reagent is rapidly agglutinated (causing light interference detected as a change in absorbance) in the presence of the anti-topiramate antibody reagent and in the absence of any competing drug in the sample. The rate of absorbance change is measured photometrically, and is directly proportional to the rate of agglutination of the particles. When a sample containing topiramate is added, the agglutination reaction is partially inhibited, slowing down the rate of absorbance change. A concentration-dependent classic agglutination inhibition curve can be obtained that is inversely proportional to topiramate concentration, with maximum rate of agglutination at the lowest topiramate concentration and the lowest agglutination rate at the highest topiramate concentration. For this microparticle turbidometric immunoassay (package insert Seradyn QMS® Topiramate), cross-reactivity to metabolite 9-hydroxytopiramate ranged from 12.5% to 22.6%.

Turbidimetric immunoassay technology described above has a limited calibration dynamic range (0-32 μg/mL). The purpose of the International Healthcontrol External Quality Assessment Scheme (EQAS) is to assess interlaboratory variability in the determination of serum levels of new antiepileptic drugs (AEDs). Participation in an EQAS scheme is recommended to ensure adequate control of assay variability in therapeutic drug monitoring (Williams J et al. Epilepsia. 2003 Jan; 44(1):40-5). Quality Control (QC) proficiency samples used by EQAS include those containing at least 40 μg/mL topiramate. In addition, commercially available topiramate QC samples (UTAK Laboratories Inc., 25020 Avenue Tibbitts, Valencia, Calif. 91355) provides the high control at a topiramate concentration of 50 μg/mL. Thus, it would be desirable to have an assay with a calibration dynamic range to minimize the need to dilute high out of range samples. More importantly, the turbidimetric immunoassay has interference from 9-hydroxytopiramate, a metabolite of topiramate. Also, the turbidimetric immunoassay has undesirable interference from phenytoin, ibuprofen, and tiagabine of greater than 10% as stated in the product insert. Since clinical scenarios indicated topiramate as adjunctive therapy given with other AEDs, it would be desirable not to have interference from other AEDs, such as phenytoin and tiagabine. Also, interference from ibuprofen, a commonly used non-steroidal anti-inflammatory which is an over-the-counter drug is highly undesirable.

Improved methods of quantifying topiramate would be an advancement in the art. This use among others is addressed by the invention described herein.

### SUMMARY OF THE INVENTION

The present invention provides for the determination of the presence or the concentration of topiramate in a sample. A variety of haptens, hapten-reactive partner conjugates, hapten derivatives, receptors, methods, and kits are useful in this determination. In one aspect, the present invention provides an improvement to the specific measurement of topiramate. Diacetonefructose derivatives find exemplary use in this respect.

In one aspect, the invention provides compounds having the structure:

T-Y-Z¹

wherein T has the structure:

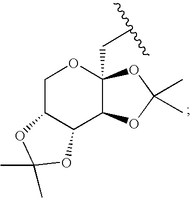

Y is a linker selected from a bond, R^{1a}, OR^{1a}, SR^{1a}, SOR^{1a}, SOOR^{1a}, SOONR^{1a}R^{1b}, NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Z¹ is a reactive functional group with the proviso that Y-Z¹ does not comprise sulfamate.

In one aspect, the invention provides compounds having the structure:

[T-Y]ᵣ—R²-Z²

wherein T has the structure:

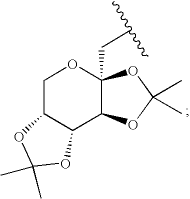

Y is a linker selected from a bond, R^{1a}, OR^{1a}, SR^{1a}, SOR^{1a}, SOOR^{1a}, SOONR^{1a}R^{1b}, NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R² is a member selected from —NHCO—, —NHCONH—, —NHCSNH—, —NHOCO—, —S—, —NH(C═NH)—, —N═N— and —NH—. Z² is a member selected from an immunogenic carrier and a signal generating moiety; and r is an integer selected from 1 to the number of T-Y binding sites on Z² with the proviso that Y-Z² does not comprise sulfamate.

In one aspect, the invention provides methods of making an antibody comprising administering to a subject a compound of the invention.

In one aspect, the invention provides antibodies generated by administering to a subject a compound of the invention.

In one aspect, the invention provides antibodies that bind topiramate and have less than about 10% cross-reactivity with a member selected from a drug and a metabolite of topiramate, wherein the drug is not topiramate.

In one aspect, the invention provides methods of determining an amount of topiramate in a sample comprising (a) contacting the sample with an antibody raised against a compound of the invention, thus yielding an antibody-topiramate complex; and (b) detecting the antibody-topiramate complex.

In one aspect, the invention provides kits for determining the amount of topiramate in a sample, the kit comprising an antibody raised against a compound of the invention and ancillary reagents.

### BRIEF DESCRIPTION OF THE DRAWINGS

FIG. 1 shows chemical structures: (1) topiramate, (2) 9-hydroxytopiramate, (3) 10-hydroxytopiramate, (4) 2,3-diol topiramate, (5) 4,5-diol topiramate, (6) diacetonefructose, and (7) 2,3:4,5-bis-O-(methylethylidene) epitope.

FIG. 2 is a flow diagram illustrating an embodiment of a method for performing an immunodiagnostic assay for topiramate. The rate of increasing absorbance at 340 nm due to the conversion of NAD⁺ (Nicotinamide adenine dinucleotide reduced) to NADH (Nicotinamide adenine dinucleotide oxidized) is related to the concentration of topiramate in the sample by a mathematical function. The enzyme reaction is catalyzed by Diacetonefructose-G6PDH (Glucose-6-phosphate dehydrogenase) conjugate.

### DETAILED DESCRIPTION OF THE INVENTION

### Definitions

The symbol [symbol] whether utilized as a bond or displayed perpendicular to a bond indicates the point at which the displayed moiety is attached to the remainder of the molecule, solid support, etc. In certain depictions of the group T, the symbol [symbol] represents the point at which T is attached to either Y or R² or Z¹ or Z².

Where substituent groups are specified by their conventional chemical formulae, written from left to right, they optionally encompass substituents resulting from writing the structure from right to left, e.g., —CH₂O— optionally also recites —OCH₂—.

The term “alkyl,” by itself or as part of another substituent, means a straight chain, branched chain, or cyclic hydrocarbon radical, or a combination thereof, which may be fully saturated, mono- or polyunsaturated and can include monovalent, divalent (i.e., an alkylene) and multivalent radicals, having the number of carbon atoms that are optionally indicated (i.e. C₁-C₁₀ means one to ten carbons). Examples of saturated hydrocarbon radicals include, but are not limited to, groups such as methyl, methylene, ethyl, ethylene, n-propyl, propylene, isopropyl, n-butyl, t-butyl, isobutyl, sec-butyl, butylene, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include but are not limited to vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl” also includes those derivatives of alkyl defined in more detail below, for example “heteroalkyl,” with the difference that the heteroalkyl group, in order to qualify as an alkyl group, is linked to the remainder of the molecule through a carbon atom. Alkyl groups that are limited to hydrocarbon groups are termed “homoalkyl.” “Lower alkyl” refers to alkyl groups having 1, 2, 3, 4, 5, 6, 7 or 8 carbon atoms. Examples of lower alkyl groups include methyl, ethyl, propyl, isopropyl, butyl, s-and t-butyl and the like.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms in exemplary embodiments. A “lower alkylene” is a short chain group, generally having 1, 2, 3, 4, 5, 6, 7 or 8 carbon atoms.

The term “alkenyl” by itself or as part of another substituent refers to a radical derived from an alkene, for example, substituted or unsubstituted vinyl and substituted or unsubstituted propenyl. Typically, an alkenyl group will have from 1 to 24 carbon atoms, with those groups having from 1 to 10 carbon atoms in exemplary embodiments.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl” by itself or in combination with another term means a straight or branched chain, or cyclic carbon-containing monovalent, divalent or multivalent radical, or combinations thereof, having any indicated number of carbon atoms and at least one heteroatom that is a member selected from the group consisting of O, N, Si, P and S, wherein the nitrogen, phosphorous and sulfur atoms are optionally oxidized, and the nitrogen heteroatom is optionally quaternized. Any of the heteroatoms O, N, P, S and Si may be placed at any interior position of the heteroalkyl group or at the position at which the alkyl group is attached to the remainder of the molecule. Examples include but are not limited to —CH₂—CH₂—O—CH₃, —CH₂—CH₂—NH—CH₃, —CH₂—CH₂—N(CH₃)—CH₃, —CH₂—S —CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —S—CH₂—CH₃, —NH—CH₂—CH₃, —S—CH₂—CH₂— and —NH—CH₂—CH₂—. Up to two heteroatoms may be consecutive, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Cycloalkyl and heterocycloalkyl groups include monovalent, divalent or multivalent radical. Additionally, for heterocycloalkyl, a heteroatom can occupy the position at which the heterocycle is attached to the remainder of the molecule. A “cycloalkyl” or “heterocycloalkyl” substituent may be attached to the remainder of the molecule directly or through a linker. A cycloalkyl or heterocycloalkyl group can be attached to the remainder of the molecule through a linkage to an atom that forms part of the cycloalkyl or heterocycloalkyl ring or through a linkage to a substituent of the cycloalkyl or heterocycloalkyl ring. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 3 rings), which are fused together or linked covalently. The term “heteroaryl” refers to aryl groups (or rings) that contain from one to four heteroatoms which are members selected from N, O, and S, wherein the nitrogen and sulfur atoms are optionally oxidized, and the nitrogen atom(s) are optionally quaternized. A heteroaryl group can be attached to the remainder of the molecule through a heteroatom. Aryl and heteroaryl groups include monovalent, divalent and multivalent radicals. An aryl or heteroaryl group can be attached to the remainder of the molecule through a linkage to an atom that forms part of the aryl or heteroaryl ring or through a linkage to a substituent of the aryl or heteroaryl ring. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems may be selected from the group of substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 8-membered ring” means there are 5, 6, 7 or 8 atoms in the encircling arrangement. The ring optionally includes at least one heteroatom. Thus, the term “5- to 8-membered ring” includes, for example, pyridinyl and piperidinyl. The term “ring” further includes a ring system comprising more than one “ring”, wherein each “ring” is independently defined as above.

Each of the above terms (e.g., alkyl, heteroalkyl, cycloalkyl, heterocycloalkyl, aryl and heteroaryl) includes both substituted and unsubstituted forms of the indicated radical. Preferred substituents for each type of radical are provided below. It will be understood that “substitution”, “substituted” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R′, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R′, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula—A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “halo” or “halogen,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” includes, but is not limited to, trifluoromethyl, 2,2,2-trifluoroethyl, 4-chlorobutyl, 3-bromopropyl, and the like.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “amino” or “amine group” refers to the group —NR′R″(or N⁺RR′R″) where R, R′ and R″ are independently selected from the group consisting of hydrogen, alkyl, substituted alkyl, aryl, substituted aryl, arylalkyl, substituted arylalkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions.

The compounds of the present invention includes salts and solvates thereof. In one embodiment, the solvate is a hydrate. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), Vogel's Encyclopedia of Practical Organic Chemistry, 5th ed., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as, for example, tritium (³H), iodine-125 (¹²⁵I) or carbon-14 (¹⁴C). All isotopic variations of the compounds of the present invention, whether radioactive or not, are intended to be encompassed within the scope of the present invention.

As used herein, a “derivative” is a compound derived or obtained from another and containing essential elements of the parent substance. Thus, in one embodiment, the term derivative refers to a chemical compound or molecule made from diacetonefructose by one or more chemical reactions wherein the derivative is not produced from a chemical reaction involving the therapeutic agent topiramate. As such, a derivative can be a compound with a structure similar to that of diacetonefructose or based on a diacetonefructose scaffold. Derivatives of diacetonefructose in accordance with the present invention can be used to compete for binding with a receptor including an antibody that recognizes both the derivative and topiramate. Also, a derivative can include an operative group coupled to diacetonefructose through a linker. Thus, the invention provides for diacetonefructose derivatives linked to, for example, an immunogenic carrier and/or a signal generating moiety as operative groups.

The term “operative group” refers to a moiety, chemical group or molecule coupled to diacetonefructose derivative through a linker. An operative group can include a reactive functional group, immunogenic carrier, signal generating moiety, antigen, tracer, solid support and the like.

The term “linker” or “linking group” refers to a portion of a chemical structure that connects two or more substructures. In some embodiments, the linker is a part of the compound of the invention. In some embodiments, the linker can provide a connection between, for example, T and R²; T and Z¹; and T and Z². The compounds of the invention may be connected to other species by bonding between a reactive functional group on the compound or a linker attached to the compound, and a reactive functional group of complementary reactivity on the other species. A linker may be, for example, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl and combinations thereof. A linker may also include cyclic and/or aromatic groups as part of the chain or as a substitution on one of the atoms in the chain. In one embodiment, the linker may be used to provide an available site on a hapten for conjugating the hapten with, for example, a label, carrier, immunogenic carrier or the like. The linker molecule may be used to connect (conjugate or couple) the ligand, hapten, epitope or epitopic moiety to its immunogenic carrier or signal generating moiety and to display the ligand, hapten, epitope or epitopic moiety for binding to the receptor or antibody. The length of the linker may be varied by those skilled in the art to accomplish the desired outcome in producing the immunogen or the signal generating system.

Reactive functional groups can be found, for example, in Hermanson, BIOCONJUGATE TECHNIQUES (Academic Press, San Diego, 1996). Reactive functional groups and classes of reactions useful in practicing the present invention are generally those that are well known in the art of bioconjugate chemistry. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982. A linker may include a reactive functional group for conjugating one structure to another. In addition, a reactive functional group can be attached to a moiety such as T.

Reaction types also include the reaction of carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters. Hydroxyl groups can be converted to esters, ethers, aldehydes and so on. Haloalkyl groups are converted to new species by reaction with, for example, an amine, a carboxylate anion, thiol anion, carbanion, or an alkoxide ion. Dienophile (e.g., maleimide) groups participate in Diels-Alder. Aldehyde or ketone groups can be converted to imines, hydrazones, semicarbazones or oximes, or via such mechanisms as Grignard addition or alkyllithium addition. Sulfonyl halides react readily with amines, for example, to form sulfonamides. Amine or sulfhydryl groups are, for example, acylated, alkylated or oxidized. Alkenes, can be converted to an array of new species using cycloadditions, acylation, Michael addition, etc. Epoxides react readily with amines and hydroxyl compounds.

Thus, in one embodiment, the reactive functional group is a member selected from amine, carboxylic acid, ester, halogen, isocyanate, isothiocyanate, thiol, thioether, thioester, imidoester, anhydride, maleimide, thiolacetone, diazonium group, aldehydes, acrylamide, acyl azide, acyl nitrile, alkyl halide, aniline, aryl halide, azide, aziridine, boronate, carboxylic acid, diazoalkane, haloacetamide, halotriazine, hydrazine, hydrazide, imido ester, phosphoramidite, reactive platinum complex, sulfonyl halide, tosylate, triflate, mesylate, imidazole and photoactivatable group.

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, Prodrugs: Topical and Ocular Drug Delivery, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 507:353-54 (1987); for disulfides, see, March supra at 1160; and for phosphonate esters and phosphonamidates.

The reactive functional groups can be chosen such that they do not participate in or interfere with the reactions necessary to assemble a reactive ligand analog (e.g. hapten). Alternatively, a reactive functional group can be protected from participating in the reaction by the presence of a protecting group. For examples of useful protecting groups, see Greene et al., Protective Groups in Organic Synthesis, John Wiley & Sons, New York, 1991.

Reactive functional groups that are used to link species may be activated. A variety of reactive functional groups, including hydroxy, amino, and carboxy groups, can be activated using a variety of standard methods and conditions. For example, a hydroxyl group of a species can be activated through treatment with phosgene to form the corresponding chloroformate, or p-nitrophenylchloroformate to form the corresponding carbonate.

The term “activated carboxyl” refers to a derivatized carboxyl group that is reactive with a biomolecule. Groups which can be used for the activation are known, and reference may be made for example to M. and A. Bodansky, “The Practice of Peptide Synthesis”, Springer Verlag 1984. Examples are adducts of the carboxylic acid with carbodiimides or activated esters such as, for example, hydroxybenzotriazole esters. Other examples include nitrophenylesters, N-hydroxysuccinimidyl esters, and those described in Chem. Soc. Rev. 12:129, 1983 and Angew. Chem. Int. Ed. Engl. 17:569, 1978.

In one embodiment, a species includes a carboxyl functionality. Carboxyl groups may be activated by, for example, conversion to the corresponding acyl halide or active ester. This reaction may be performed under a variety of conditions as illustrated in March, supra pp. 388-89. In one embodiment, the acyl halide is prepared through the reaction of the carboxyl-containing group with oxalyl chloride.

In one embodiment, compounds can be prepared by reaction of an amino-containing intermediate with an activated carboxyl- or sulfonyl-containing reagent in the presence of an appropriate base (e.g. TEA, DIEA, N-methylmorpholine, pyridine, DMAP, or the like), as needed. Suitable carboxyl- or sulfonyl-containing reagents include, but are not limited to, acid chlorides, acid fluorides, sulfonyl chlorides, sulfonyl fluorides, polystyrene-2,3,5,6-tetrafluoro-4-(methylcarbamoyl)phenol (PS-TFP)-carboxylates, PS-TFP-sulfonates, carbamoyl chlorides, isocyanates, isothiocyanates, anhydrides, chloroformates, HOBt ester, carbodiimide-derived O-acylurea, and the like.

The term “hydroxyl activating group” refers to group that replaces the hydrogen of the hydroxyl group, thereby altering the chemical and electronic properties of the hydroxyl group such that the hydroxyl group is more susceptible to removal, such as by replacement with hydrogen or a moiety other than a hydroxyl group. An “activated hydroxyl group” thus refers to the structure —OR′ wherein R′ is a hydroxyl activating group. Exemplary hydroxyl activating groups include, for example, alkyl, aryl, aralkyl, heteroaryl, heterocyclyl, alkylcarbonyl, arylcarbonyl, aralkylcarbonyl, heteroarylcarbonyl, heterocyclylcarbonyl, C(S)O-aryl, C(S)O-alkyl, or silyl. Examples of activated hydroxyl groups include tosyl, triflate, mesylate and —O-imidazole.

The term “hapten” refers to small molecular weight compounds (e.g., reactive ligand, epitope, epitopic moiety, antigenic moiety or immunogenic moiety) that bind specifically to corresponding antibodies but usually do not themselves act as complete immunogens for preparation of the antibodies. Antibodies that recognize a hapten can be prepared against compounds comprising the hapten linked to an immunogenic carrier, together referred to as an immunogen. A hapten is usually non-immunogenic alone due to its low molecular weight and when linked to a large molecular weight carrier (e.g., protein) the hapten comprises the immunogenic moiety of interest in producing antibodies in the immunized subject or animal. In an exemplary embodiment, the hapten is a diacetonefructose derivative.

The term “label”, “label moiety” or “signal generating moiety” refers to any molecule that produces or can be induced to produce a detectable signal. Non-limiting examples of a signal generating moiety include radioactive isotopes, synthetic polymers, saccharides (including monosaccharides and polysaccharides), peptides (including single amino acids and poly(amino acids), i.e., polypeptides), proteins, enzymes, enzyme fragments, enzyme substrates, enzyme inhibitors, coenzymes, catalysts, fluorophores, dyes, chemiluminescers, luminescers, sensitizers, non-magnetic or magnetic particles, solid supports, liposomes, ligands, receptors, hapten radioactive isotopes, and the like. In one embodiment, the signal generating moiety is a member selected from glucose-6-phosphate dehydrogenase (G6PDH), alkaline phosphatase, B-galactosidase and horseradish peroxidase. As used herein, the terms “protein” and “polypeptide” are synonymous.

The signal generating moiety can be conjugated either directly or through a linker to a diacetonefructose derivative, hapten, analyte, immunogen, antibody, or to another molecule such as a receptor or a molecule that can bind to a receptor. As described herein, the derivatives or haptens can also be coupled to a variety of signal generating moieties by methods well known in the art to provide a variety of reagents useful in various immunoassay formats. For detecting the results of the immunoassays, detector molecules such as fluorophores (for example, fluorescein), radiolabels, or chemiluminescent groups can be coupled to haptens to produce tracers. The term “tracer” refers to a compound that is coupled to a signal generating moiety.

The term “antibody” refers to a protein molecule having one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), epsilon (ε), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. Thus, the term “antibody raised against a compound” includes a synthesized antibody or compound having the same structure as an antibody raised against the compound The term “antibody” includes antibody fragments, as are known in the art, such as Fab, Fab′, F(ab′)₂, Fv, scFv, or other antigen-binding subsequences of antibodies, either produced by the modification of whole antibodies or those synthesized de novo using recombinant DNA technologies. The term “antibody” refers to both monoclonal and polyclonal antibodies. Antibodies can be antagonists, agonists, neutralizing, inhibitory, or stimulatory.

As used herein, the term “polyclonal antibody” refers to a heterogeneous mixture of antibodies with a wide range of specificities and affinities to a given antigen or epitope. Thus, the polyclonal antibody, which can also be referred to as polyclonal antibodies, can include a plurality of antibodies, each distinguishable from the others, that bind or otherwise interact with an antigen. The term “polyclonal” refers to antibodies originating from multiple progenitor cells. The different antibodies that comprise a polyclonal antibody can be produced or generated by injecting an immunogen having an epitope into an animal and, after an appropriate time, collecting and optionally purifying the blood fraction containing the antibodies of interest. In producing antibodies, several parameters can be considered with respect to the final use for the polyclonal antibody. These parameters include the following: (1) the specificity of the antibody (i.e., the ability to distinguish between antigens); (2) the avidity of the antibody (i.e., the strength of binding an epitope); and (3) the titer of the antibody, which determines the optimal dilution of the antibody in the assay system.

As used herein, the term “monoclonal antibody” refers to an antibody that is isolated from a culture of normal antibody-producing cells and one unique progenitor cell. A monoclonal antibody can have a homogeneous binding constant.

The antibodies of the present invention may be nonhuman, chimeric, humanized, or fully human. For a description of the concepts of chimeric and humanized antibodies see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see for example U.S. Pat. No. 4,816,567). In a one embodiment, the antibodies of the present invention are humanized. By “humanized” antibody as used herein is meant an antibody comprising a human framework region (FR) and one or more complementarity determining regions (CDRs) from a nonhuman (for example, mouse or rat) antibody. The nonhuman antibody providing the CDRs is called the “donor” and the human immunoglobulin providing the framework is called the “acceptor”. Humanization relies principally on the grafting of donor CDRs onto acceptor (human) VL and VH frameworks (U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). Methods for humanizing nonhuman antibodies are well known in the art, and can be essentially performed following the method of Winter and co-workers (Jones et al., 1986, Nature 321:522-525; Riechmann et al., 1988, Nature 332:323-329; Verhoeyen et al., 1988, Science, 239:1534-1536). Additional examples of humanized murine monoclonal antibodies are also known in the art, for example antibodies binding human protein C (O'Connor et al., 1998, Protein Eng 11:321-8), interleukin 2 receptor (Queen et al., 1989, Proc Natl Acad Sci, USA 86:10029-33), and human epidermal growth factor receptor 2 (Carter et al., 1992, Proc Natl Acad Sci USA 89:4285-9). In one embodiment, the antibodies of the present invention may be fully human, that is, the sequences of the antibodies are completely or substantially human. A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

The term “inhibitory antibody” refers to an antibody capable of inhibiting the activity of an enzyme or an enzyme-ligand conjugate upon binding an epitope present on the enzyme. Such antibodies are distinguished from anti-ligand antibodies capable of inhibiting the enzyme activity of enzyme-ligand conjugates upon binding to the ligand.

“Antigen”, as used herein, refers to a compound that binds specifically to the variable region or binding site of an antibody. The term “antigen” and “immunogen” may in some cases be used interchangeably.

The term “epitope” refers to a region of an antigen that interacts with an antibody molecule. An antigenic molecule can have one or more epitopes that can be recognized by the same or different antibodies. An epitope or epitopic moiety may comprise a unique chemical configuration of an antigen, hapten or a reactive ligand. The chemical configuration may be a linear sequence of chemical composition or even a spatial array of chemical groups in the chemical configuration. An epitope is the chemical configuration that associates directly with the binding site in the antibody molecule. The antibody and the chemical group, hapten or reacting ligand containing the epitope form the “specific binding pair”.

As used herein, the terms “immunogen” and “immunogenic” are meant to refer to substances capable of producing or generating an immune response (e.g., antibody response) in an organism. An immunogen can also be antigen. In one embodiment, the immunogen has a fairly high molecular weight (e.g. greater than 10,000). Thus, a variety of macromolecules such as proteins, lipoproteins, polysaccharides, nucleic acids and teichoic acids can be coupled to a hapten in order to form an immunogen in accordance with the present invention.

As used herein, the term “immunogenicity” refers to the ability of a molecule to induce an immune response, which is determined both by the intrinsic chemical structure of the injected molecule and by whether or not the host animal can recognize the compound. Small changes in the structure of an antigen can greatly alter the immunogenicity of a compound and have been used extensively as a general procedure to increase the chances of raising an antibody, particularly against well-conserved antigens. For example, these modification techniques either alter regions of the immunogen to provide better sites for T-Cell binding or expose new epitopes for B-cell binding.

“Immunogenic carrier”, “carrier,” or “immunogenic moiety,” as used herein, refers to any material that when combined with a hapten stimulates an in vitro or in vivo immune response. A hapten becomes an immunogenic moiety when coupled to a carrier and as part of the immunogen can induce an immune response and elicit the production of antibodies that can bind specifically with the hapten. Immunogenic carrier moieties include proteins, peptides (including polypeptides), glycoproteins, saccharides including complex polysaccharides, particles, nucleic acids, polynucleotides, and the like that are recognized as foreign and thereby elicit an immunologic response from the host. Additionally, linkers that link a carrier to a hapten can comprise modified or unmodified nucleotides, nucleosides, polymers, sugars and other carbohydrates, polyethers, such as for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes. An example of an operative group and its linker is cholesterol-TEG-phosphoramidite, wherein the cholesterol is the operative group and the tetraethylene glycol and phosphate serve as linkers.

In one embodiment, the immunogenic carrier is a protein. Protein carriers can be highly soluble and include functional groups that could facilitate easy conjugation with a hapten molecule. In an exemplary embodiment, the immunogenic carrier is a member selected from keyhole limpet hemocyanin (KLH), bovine serum albumin (BSA) and human serum albumin (HSA). Keyhole limpet hemocyanin is an oxygen-carrying protein of the marine keyhole limpet, is extremely large and exhibits increased immunogenicity when it is disassociated into subunits. BSA is a highly soluble protein containing numerous functional groups suitable for conjugation.

A “ligand” refers to a compound for which a receptor naturally exists or can be prepared. A “receptor” refers to a binding partner of a ligand. In one embodiment, a diacetonefructose derivative is a ligand that is bound to a receptor such as an antibody. In one embodiment, the ligand is a diacetonefructose derivative conjugated to an operative group such as an immunogenic carrier or a signal-generating moiety.

“Conjugate”, as used herein, refers to a molecule comprising two or more moieties bound together, optionally through a linker or linking group, to form a single structure. The binding can be made either by a direct connection (e.g. a chemical bond) between the subunits or by use of a linking group. Examples and methods of forming conjugates are further described in Hermanson, G. T., “Bioconjugate Techniques”, Academic Press: New York, 1996; and “Chemistry of Protein Conjugation and Cross-linking” by S. S. Wong, CRC Press, 1993, herein incorporated by reference. In one embodiment, the conjugate is a G6PDH enzyme or a label protein such as alkaline phosphatase, B-galactosidase and horse radish peroxidase or a chemical label such as a fluorescent, luminescent or colorimetric molecule attached to a hapten, specific binding pair member, reactive ligand or analyte.

“Homogeneous immunoassay”, as used herein, refers to an assay method where the complex is typically in solution and not separated from unreacted reaction components, but instead the presence of the complex is detected by a property which at least one of the reactants acquires or loses as a result of being incorporated into the complex. Homogeneous assays known in the art include systems involving fluorochrome and fluorochrome quenching pairs on different reagents (U.S. Pat. Nos. 3,996,345; 4,161,515; 4,256,834 and 4,264,968); enzyme and enzyme inhibitor pairs on different reagents (U.S. Pat. Nos. 4,208,479 and 4,233,401); chromophore and chromophore modifier pairs on different reagents (U.S. Pat. No. 4,208,479); and latex agglutination assays (U.S. Pat. Nos. 3,088,875; 3,551,555; 4,205,954 and 4,351,824).

“Human serum”, as used herein, refers to the aqueous portion of human blood remaining after the fibrin and suspended material (such as cells) have been removed.

“Buffered synthetic matrix”, as used herein, refers to an aqueous solution comprising non-human constituents. Buffered synthetic matrices may include surface active additives, organic solvents, defoamers, buffers, surfactants, and anti-microbial agents. Surface active additives are introduced to maintain hydrophobic or low-solubility compounds in solution, and stabilize matrix components. Examples include bulking agents such as betalactoglobulin (BLG) or polyethyleneglycol (PEG); defoamers and surfactants such as Tween-20, Plurafac A38, Triton X-100, Pluronic 25R2, rabbit serum albumin (RSA), bovine serum albumin (BSA), and carbohydrates. Examples of organic solvents in buffered synthetic matrices include methanol and other alcohols. Various buffers may be used to maintain the pH of the synthetic matrix during storage. Illustrative buffers include HEPES, borate, phosphate, carbonate, tris, barbital and the like. Anti-microbial agents also extend the storage life of the matrix. An example of an anti-microbial agent used in this invention includes 2-methyl-4-isothiazolin-3-one hydrochloride.

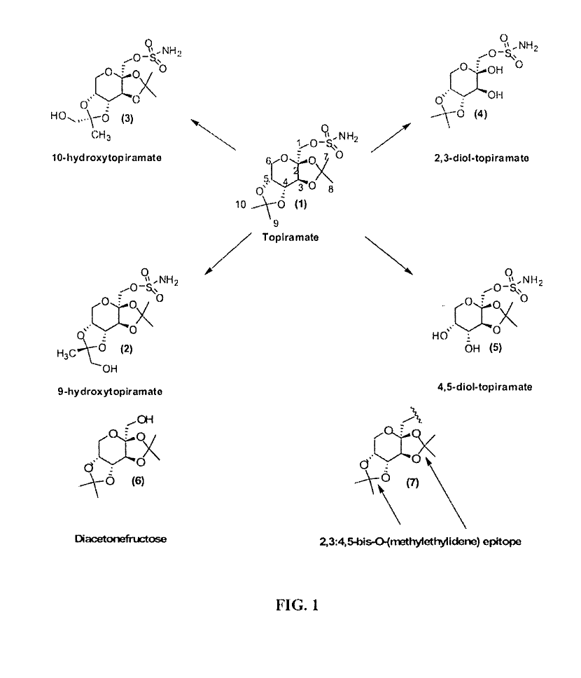

As used herein, the term “affinity” refers to a measure of the strength of binding between an epitope and an antibody. Accordingly, a single antibody can have a different affinity for various epitopes. This can allow a single antibody to bind strongly to one epitope and less strongly to another. As such, an antibody can have a first affinity to a drug, such as topiramate, and have a second affinity to a diacetonefructose derivative. However, it is possible for the antibody to have substantially equivalent or similar affinity for both topiramate and a diacetonefructose derivative, which allows the derivative to be used to generate antibodies for topiramate, and their use in competitive binding studies. Thus, diacetonefructose derivatives in accordance with the present invention can be used to generate antibodies with affinity for topiramate. It is noted in FIG. 1 that Structure 6 (diacetonefructose) and Structure 1 (topiramate) both comprise the epitopic moiety Structure 7 (2,3:4,5-bis-O-(methylethylidene)) that is of interest in producing antibodies that are highly selective for measurement of topiramate. It is further noted that the sulfamate moiety present in the topiramate structure is absent in diacetonefructose derivatives which contributes to the antibody specificity.

The term “precision” refers to the closeness of agreement between independent test/measurement results obtained under stipulated conditions. Coefficient of variation (CV %) is a statistical measurement of the relative variation (dispersion) of data points of a sample based on a 100% scale. The formulation for CV, which is expressed in a percentage, is given by

CV %=(SD÷Mean)×100.

The term “accuracy” refers to the closeness of the agreement between the result of a measurand and a true value of the measurand. The measurand is the substance measured or analyzed, the analyte or the ligand entering the binding reaction with the receptor or antibody.

The term “specificity” or “selectivity” refers to the preferential binding of a ligand to a receptor (e.g., antibody). Thus, specificity may refer, in one embodiment, to the degree that topiramate is bound selectively by an antibody. One measure of the specificity of a receptor to a ligand is crossreactivity. Compounds that cross-react are referred to as “crossreactants.” “Crossreaction” is the non-specific binding of crossreactants to receptors or antibodies due to the chemical similarity of the cross-reacting compound or a moiety of the cross-reacting compound to the epitopic moiety of a hapten, ligand or target analyte of interest. In one embodiment, the target analyte of interest is topiramate and the crossreactant is a drug that is not topiramate. The drug that is not topiramate may include various types of drugs administered to treat various types of conditions. These drugs include antiepileptic drugs, antiinflammatory drugs, anticonvulsive drugs and antibacterial sulfonamides. In an exemplary embodiment, the antiepileptic drug is phenytoin, the antiinflammatory drug is ibuprofen and the anticonvulsive drug is tiagabine.

In an exemplary embodiment, anti-diacetonefructose derivative antibodies are highly selective for measurement of topiramate and do not crossreact significantly with a drug or metabolites of topiramate, wherein the drug is not topiramate. In this embodiment, diacetonefructose derivatives comprise the epitope also present in topiramate and antibodies specific for this shared epitope avoid reaction with a sulfamate moiety that may be present in other drugs. Also, metabolism of topiramate may cause chemical changes to this epitopic moiety. These chemical changes may lower the avidity of anti-diacetonefructose derivative antibodies for binding to metabolites of topiramate, which corresponds to lower crossreactivity to metabolites of topiramate. It can be seen in FIG. 1 that Structure 2 (9-hydroxytopiramate), Structure 3 (10-hydroxytopiramate), Structure 4 (2,3-diol topiramate) and Structure 5 (4,5-diol topiramate) are metabolites of topiramate comprised of chemical changes to the epitopic moiety Structure 7 (2,3:4,5-bis-O-(methylethylidene)).

In one aspect, the present invention provides for receptors (e.g., antibodies) that have less than about 10% cross-reactivity with a drug and/or a metabolite of topiramate, wherein the drug is not topiramate. In one embodiment the receptor has about 9%, about 8%, about 7%, about 6%, about 5%, about 4%, about 3%, about 2% or about 1% cross-reactivity with a drug and/or a metabolite of topiramate, wherein the drug is not topiramate. In one embodiment, the receptor is an antibody with high specificity for the 2,3:4,5-bis-O-(methylethylidene) moiety of topiramate and will not significantly cross-react with topiramate metabolites or with a drug such as phenytoin, ibuprofen, and tiagabine, or other sulfamate or sulfonamide moiety containing compounds. By “not significantly” is meant a cross-reactivity of less than about 10%, less than about 5%, or less than about 1%. Measuring the degree of cross-reactivity may demonstrate the capacity of a receptor or antibody to measure, detect or identify a target substance, ligand or analyte selectively. The absence of cross-reactivity implies a high degree of specificity for the target analyte or ligand to be measured, detected or identified. The use of highly specific or selective antibodies in an assay is a contributing factor for high accuracy of measurement. The percentage crossreactivity may be calculated by the following formula:

% Crossreactivity=100×(“apparent concentration of measurand”/“concentration of crossreactant”)

The term “interference” refers to the effect of a substance present in the sample that alters the correct value of a result, usually expressed as concentration or activity or percentage, for a substance. The major exogenous sources of interference are drugs prescribed for the patient and their metabolites. Interference may also be caused by endogenous substances in serum or plasma (e.g., lipids, bilirubin and hemoglobin). The absence of interference also implies a high degree of specificity and accuracy by the test system for the target analyte or ligand to be measured, detected or identified. The percentage interference may be calculated by the following formula:

% Interference=100×(“False result”−“True result”)/(“True result”)

As used herein, the terms “immunoassay” or “immunodiagnostic” refer to laboratory techniques or test systems that make use of the binding between an antigen and an antibody in order to identify and/or quantify at least one of the specific antigen or specific antibody in a biological sample. Currently, there are three classes of immunoassay, which are described as follows: (1) antibody capture assays; (2) antigen capture assays; and (3) two-antibody sandwich assays. Additionally, it is contemplated that new immunoassays will be developed and will be capable of employing the hapten derivatives and antibodies that form the specific binding pair of the present invention. Immunoassay or immunodiagnostic test systems measure a ligand or target analyte, the measurand (e.g., topiramate), by using the selective binding properties of an antibody and a signal generating system comprising a signal generating moiety that is responsive or reactive to the presence of antibody due to the binding of the antibody with hapten conjugated to the signal generating moiety.

As used here, the term “competitive immunoassay” refers to a experimental protocol in which a known amount of an identifiable antigen competes with another antigen for binding with an antibody. That is, a known antigen that binds with a known antibody is combined with a sample that is suspected of containing another antigen that also binds with the known antibody. This allows for the known antigen and another antigen to both compete for the binding site on the antibody. For example, a diacetonefructose derivative that binds with an anti-diacetonefructose derivative antibody can be combined with a sample suspected of containing topiramate, and the derivative and topiramate compete for binding with the anti-diacetonefructose derivative antibody. The competition for binding with the antibody can then be used to determine whether or not topiramate is present in the sample, and can further be used to quantify the amount of topiramate in the sample.

Homogeneous enzyme immunoassays depend on the availability of enzyme-hapten conjugates whose enzyme activity can be strongly modulated upon binding of an antibody raised against an epitope present on the hapten. In one aspect, the present invention provides enzyme-hapten conjugates and antibodies for conducting assays that are useful in homogeneous immunoassays.

The anti-diacetonefructose derivative antibodies, either monoclonal or polyclonal, can be used in immunoassays for identifying the presence of topiramate in a biological sample, such as blood, plasma, serum, urine, tissue, and the like. This can be beneficial for identifying or determining pharmacokinetic and/or pharmacodynamic parameters for topiramate in a patient or patient population. Thus, the anti-diacetonefructose derivative antibodies can be used in immunodiagnostic assays so that the assays can be configured for identifying the presence and optionally quantifying the amount of topiramate. Additionally, the immunodiagnostic assays can use diacetonefructose derivatives in accordance with the present invention.

### Compounds of the Invention

In one aspect, the invention provides any of the compounds disclosed herein.

### Diacetonefructose Derivatives

In one aspect, the invention provides diacetonefructose derivatives. These derivatives that are useful as haptens may be used as immunogens with linkage to an immunogenic carrier to induce an immune response in a subject or animal to generate antibodies. Diacetonefructose derivates may also be conjugated, for example to a signal generating moiety for use in immunoassays.

Therefore, in one aspect, the invention provides compounds having the structure:

T-Y-Z¹

wherein T has the structure:

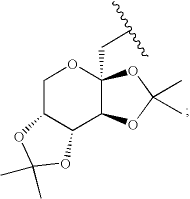

Y is a linker selected from a bond, R^{1a}, OR^{1a}, SR^{1a}, SOR^{1a}, SOOR^{1a}, SOONR^{1a}R^{1b}, NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Z¹ is a reactive functional group, with the proviso that Y-Z¹ does not comprise sulfamate.

In one embodiment, the compound has the structure:

T-Y-Z¹

wherein T has the structure:

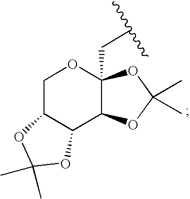

Y is a linker selected from a bond, R^{1a}, OR^{1a}, NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Z¹ is a reactive functional group, with the proviso that Y-Z¹ does not comprise sulfamate.

In one embodiment, when Y is SOONR^{1a}R^{1b} or NR^{1a}R^{1b}, Z¹ is covalently attached to Y through R^{1a}.

In one embodiment, Z¹ is covalently attached to Y through R^{1a}.

In an exemplary embodiment, the reactive functional group is a member selected from an electrophilic group and a nucleophilic group.

In an exemplary embodiment, the electrophilic group is a member selected from activated ester, acyl azide, acyl halide, acyl nitrile, aldehyde, alkyl halide, alkyl sulfonate, anhydrides, aryl halide, aziridine, boronate, caroxylic acid, carbodiimides, diazoalkane, epoxide, haloacetamide, halotriazine, imidoester, isocyanate, isothiocyanate, ketone, maleimide, phosphoramidite, silyl halide, sulfonate ester and sulfonyl halide.

In an exemplary embodiment, the nucleophilic group is a member selected from alcohol, amine, aniline, carboxylic acid, glycol, hydrazine, hydroxylamine, phenol and thiol.

In an exemplary embodiment, the reactive functional group is a member selected from amine, ester, thioester, thioether, halogen, isocyanate, isothiocyanate, thiol, imidoester, anhydride, maleimide, thiolacetone, diazonium groups, aldehyde, succinimide, hydroxysuccinimide, imidate, tosylate, triflate, mesylate and imidazole.

In one embodiment, Y-Z¹ or Y—R²-Z² does not comprise a member selected from sulfonamide, sulfonyl and sulfidyl.

In an exemplary embodiment, Y does not comprise sulfamate.

In an exemplary embodiment, Z¹ does not comprise sulfamate.

In an exemplary embodiment, Y does not comprise a sulfur atom.

In an exemplary embodiment, Z¹ does not comprise a sulfur atom.

In one embodiment, the diacetonefructose derivative comprises a linker comprising about 1, 2, 3, 4, 5, 6, 7, 8, 9, 10, 11, 12, 13, 14 or 15 atoms and comprises a backbone of 2, 3, 4, 5, 6, 7 or 8 atoms, each independently selected from the group normally consisting of carbon, oxygen, sulfur, nitrogen, halogen and phosphorous. In some embodiments the linker comprises 1, 2, 3, 4, 5, 6, 7, 8, 9, 10, 11, 12, 13, 14 or 15 carbon atoms and 0, 1, 2, 3, 4, 5 or 6 heteroatoms. The length of the linker may be varied by those skilled in the art to accomplish the desired outcome in producing the immunogen or the signal generating system. Where the linker provides attachment of a protein to the modified hydroxyl group of diacetonefructose, the linker comprises at least 5 atoms or, when fewer than 5 atoms, the linker does not comprise solely carbon atoms or oxygen atoms. Examples of linker include —(CH₂)ₙC(O)—, —(CH₂)ₙC(—SO₂)═CH₂, —C(O)(CH₂)ₙ—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ— and —(CH₂)ₙNHC(O)— wherein n is an integer selected from 1, 2, 3, 4, 5, 6, 7, 8, 9 and 10.

In an exemplary embodiment, the linker comprises 0, 1, 2, 3, 4, 5 or 6 heteroatoms. The linkers may be aliphatic or aromatic. When heteroatoms are present, oxygen may be present as oxo or oxy, bonded to carbon, sulfur, nitrogen or phosphorous. Nitrogen may be present as nitro, nitroso or amino, bonded to carbon, oxygen, sulfur or phosphorous. Sulfur forms would be analogous to oxygen. Phosphorous may be bonded to carbon, sulfur, oxygen or nitrogen, sometimes as phosphonate and phosphate mono- or diester. Common reactive functional groups in forming a covalent bond between the linker and the molecule to be conjugated include alkylamine, amidine, thioamide, ether, urea, thiourea, guanidine, azo, thioether and carboxylate, sulfonate, and phosphate esters, amides and thioesters.

In an exemplary embodiment, when a linker comprises a non-oxocarbonyl group including nitrogen and sulfur analogs, a phosphate group, an amino group, alkylating agent such as halo or tosylalkyl, oxy (hydroxyl or the sulfur analog, mercapto) oxocarbonyl (e.g., aldehyde or ketone), or active olefin such as a vinyl sulfone or α-, β-unsaturated ester, these reactive functional groups will be linked to amine groups, carboxyl groups, active olefins, alkylating agents, e.g., bromoacetyl. Where an amine and carboxylic acid or its nitrogen derivative or phosphoric acid is linked, amides, amidines and phosphoramides will be formed. Where mercaptan and activated olefin are linked, thioethers will be formed. Where a mercaptan and an alkylating agent are linked, thioethers will be formed. Where aldehyde and an amine are linked under reducing conditions, an alkylamine will be formed. Where a carboxylic acid or phosphate acid and an alcohol are linked, esters will be formed. Various linking agents are well known in the art; see, for example, Cautrecasas, J. Biol. Chem. (1970) 245:3059.

Thus, in one embodiment, Y comprises a backbone of 2-8 atoms that are members independently selected from C, O, S, N, P and halogen.

In one embodiment Y has the structure

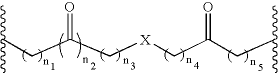

wherein each n₁, each n₃, each n₄ and each n₅ is independently selected from 0 to 10; n₂ is an integer selected from 0 and 1; and X is a member selected from S, O, NR³ and a bond wherein R³ is a member selected from H and substituted or unsubstituted alkyl.

In one embodiment, Y is a member selected from —(CH₂)ₙC(O)—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, and —(CH₂)ₙNHC(O)—; and n is an integer selected from 0, 1, 2, 3, 4, 5, 6, 7, 8, 9 and 10. In one embodiment, n is an integer selected from 1 and 2.

In one embodiment, Y is a bond. Thus, in one embodiment, the compound has the structure T-Z¹.

In one embodiment, Y-Z¹ is NH₂.

In one embodiment, Y-Z¹ is a activated hydroxyl group selected from tosylate, triflate, mesylate and imidazole.

In one embodiment, Y is —NR⁴R⁵—wherein R⁴ is selected from H, substituted or unsubstituted alkyl and substituted or unsubstituted heteroalkyl; and R⁵ is selected from substituted or unsubstituted alkyl and substituted or unsubstituted heteroalkyl. In an exemplary embodiment, Y is a member selected from —NHCOCH₂—, —NHCO(CH₂)₂—, —NHCO(CH₂)₂CONH(CH₂)₅—, —NHCOCH₂SCH₂— and —N(CH₃)CH₂—.

In one embodiment, Z¹ is a member selected from —COOR⁴ and —SR⁵, wherein R⁴ and R⁵ are members each independently selected from H, halogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In an exemplary embodiment, R⁴is a member selected from H, Br, succinimidyl and phenylmethyl; and R⁵ is a member selected from H and —COCH₃.

### Diacetonefructose Derivative Conjugates

The present invention further provides conjugates and complexes of diacetonefructose derivatives. The invention thus provides for diacetonefructose derivatives that may be linked to a conjugate via a linker. In one embodiment, the conjugate comprises an operative group.

In one aspect, the invention provides compounds having the structure:

[T-Y]ᵣ—R²-Z²

wherein T has the structure:

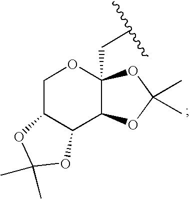

Y is a linker selected from a bond, R^{1a}, OR^{1a}, SR^{1a}, SOR^{1a}, SOOR^{1a}, SOONR^{1a}R^{1b}, NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R² is a member selected from —NHCO—, —NHCONH—, —NHCSNH—, —NHOCO—, —S—, —NH(C═NH)—, —N═N— and —NH—. Z² is a member selected from an immunogenic carrier and a signal generating moiety; and r is an integer selected from 1 to the number of T-Y binding sites on Z² with the proviso that Y-Z² does not comprise sulfamate.

In one embodiment, the compound has the structure:

[T-Y]ᵣ—R²-Z²

wherein T has the structure:

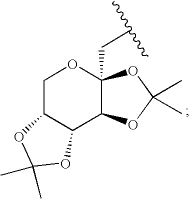

Y is a linker selected from a bond, R^{1a}, OR^{1a}NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R² is a member selected from —NHCO—, —NHCONH—, —NHOCO—, —NH(C═NH)—, —N═N— and —NH—. Z² is a member selected from an immunogenic carrier and a signal generating moiety; and r is an integer selected from 1 to the number of T-Y binding sites on Z² with the proviso that Y-Z² does not comprise sulfamate.

In one embodiment, Y-Z¹ or Y—R²-Z² does not comprise a member selected from sulfonamide, sulfonyl and sulfidyl.

In an exemplary embodiment, Y does not comprise sulfamate.

In an exemplary embodiment, Z² does not comprise sulfamate.

In an exemplary embodiment, R² does not comprise sulfamate.

In an exemplary embodiment, Y does not comprise a sulfur atom.

In an exemplary embodiment, Z² does not comprise a sulfur atom.

In an exemplary embodiment, R² does not comprise a sulfur atom.

In one embodiment, Y is a bond. Thus, in one embodiment, the compound has the structure Tᵣ-R²-Z².

In one embodiment, the diacetonefructose derivative comprises a linker comprising about 1, 2, 3, 4, 5, 6, 7, 8, 9, 10, 11, 12, 13, 14 or 15 atoms and comprises a backbone of 2, 3, 4, 5, 6, 7 or 8 atoms, each independently selected from the group normally consisting of carbon, oxygen, sulfur, nitrogen, halogen and phosphorous. In some embodiments the linker comprises 1, 2, 3, 4, 5, 6, 7, 8, 9, 10, 11, 12, 13, 14 or 15 carbon atoms and 0, 1, 2, 3, 4, 5 or 6 heteroatoms. The length of the linker may be varied by those skilled in the art to accomplish the desired outcome in producing the immunogen or the signal generating system. Where the linker provides attachment of a protein to the modified hydroxyl group of diacetonefructose, the linker comprises at least 5 atoms or, when fewer than 5 atoms, the linker does not comprise solely carbon atoms or oxygen atoms. Examples of linker include —(CH₂),C(O)—, —(CH₂)ₙC(—SO₂)═CH₂, —C(O)(CH₂)ₙ, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ— and —(CH₂)ₙNHC(O)— wherein n is an integer selected from 1, 2, 3, 4, 5, 6, 7, 8, 9 and 10.

In an exemplary embodiment, the linker comprises 0, 1, 2, 3, 4, 5 or 6 heteroatoms. The linker may be aliphatic or aromatic. When heteroatoms are present, oxygen may be present as oxo or oxy, bonded to carbon, sulfur, nitrogen or phosphorous. Nitrogen may be present as nitro, nitroso or amino, bonded to carbon, oxygen, sulfur or phosphorous. Sulfur forms would be analogous to oxygen. Phosphorous may be bonded to carbon, sulfur, oxygen or nitrogen, sometimes as phosphonate and phosphate mono- or diester. Common reactive functional groups in forming a covalent bond between the linker and the molecule to be conjugated include alkylamine, amidine, thioamide, ether, urea, thiourea, guanidine, azo, thioether and carboxylate, sulfonate, and phosphate esters, amides and thioesters.

In an exemplary embodiment, when a linker comprises a non-oxocarbonyl group including nitrogen and sulfur analogs, a phosphate group, an amino group, alkylating agent such as halo or tosylalkyl, oxy (hydroxyl or the sulfur analog, mercapto) oxocarbonyl (e.g., aldehyde or ketone), or active olefin such as a vinyl sulfone or α-, β-unsaturated ester, these reactive functional groups will be linked to amine groups, carboxyl groups, active olefins, alkylating agents, e.g., bromoacetyl. Where an amine and carboxylic acid or its nitrogen derivative or phosphoric acid is linked, amides, amidines and phosphoramides will be formed. Where mercaptan and activated olefin are linked, thioethers will be formed. Where a mercaptan and an alkylating agent are linked, thioethers will be formed. Where aldehyde and an amine are linked under reducing conditions, an alkylamine will be formed. Where a carboxylic acid or phosphate acid and an alcohol are linked, esters will be formed. Various linking agents are well known in the art; see, for example, Cautrecasas, J. Biol. Chem. (1970) 245:3059.

Thus, in one embodiment, Y comprises a backbone of 2-8 atoms that are members independently selected from C, O, S, N, P and halogen.

In one embodiment, Y has the structure

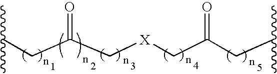

wherein each n₁, each n₃, each n₄ and each n₅ is independently selected from 0 to 10; n₂ is an integer selected from 0 and 1; and X is a member selected from S, O, NR³ and a bond wherein R³ is a member selected from H and substituted or unsubstituted alkyl.

In one embodiment, Y is a member selected from —(CH₂)ₙC(O)—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, and —(CH₂)ₙNHC(O)—; and n is an integer selected from 0, 1, 2, 3, 4, 5, 6, 7, 8, 9 and 10. In an exemplary embodiment, n is an integer selected from 1 and 2.

In one embodiment, Y is —NR⁴R⁵—wherein R⁴ is selected from H, substituted or unsubstituted alkyl and substituted or unsubstituted heteroalkyl; and R⁵ is selected from substituted or unsubstituted alkyl and substituted or unsubstituted heteroalkyl.

In an exemplary embodiment, Y is a member selected from —NHCOCH₂—, —NHCO(CH₂)₂—, —NHCO(CH₂)₂CONH(CH₂)₅—, —NHCOCH₂SCH₂— and —N(CH₃)CH₂—.

In an exemplary embodiment, R² is selected from —CONH— and —S—; and Z² is a member selected from KLH and G6PDH.

In one embodiment, the immunogenic carrier is a member selected from hemocyanin, globulin, albumin, and polysaccharide. In an exemplary embodiment, the albumin is a member selected from bovine serum albumin (BSA) and human serum albumin (HSA) and the hemocyanin is keyhole limpet hemocyanin (KLH).

In one embodiment, the signal generating moiety is a member selected from a polypeptide, a polysaccharide, a synthetic polymer, an enzyme, a fluorogenic compound and a chemiluminescent compound. In one embodiment, the enzyme is a member selected from dehydrogenase, phosphatase, galactosidase and peroxidase. In an exemplary embodiment, the enzyme is a member selected from glucose-6-phosphate dehydrogenase (G6PDH), alkaline phosphatase, B-galactosidase and horseradish peroxidase.

The diacetonefructose derivatives, bio-conjugates, antibodies, immunogens, and other conjugates described herein are also suitable for any of a number of other heterogeneous immunoassays with a range of detection systems including but not limited to enzymatic or fluorescent systems, and/or homogeneous immunoassays including but not limited to rapid lateral flow assays and antibody arrays.

While various immunodiagnostic assays have been described herein that utilize the diacetonefructose derivatives, bio-conjugates, antibodies, immunogens and/or labels, such assays can also be modified as is well known in the art. As such, various modifications of steps or acts for performing such immunoassays can be made within the scope of the present invention.

For example, an operative group including immunogenic carriers and signal generating moieties may be derivatized to facilitate attachment to a diacetonefructose derivative. For example, any operative group, e.g., KLH or G6PDH, may comprise —NH₂, —SH, —NHCO(CH₂)₂COOH, —NHCO(CH₂)₅NH₂, —NHCOCH₂Br, and —NHCOCH₂SH.

Immunogens comprising, for example, proteins are synthesized and used to prepare antibodies specific for the 2,3:4,5-bis-O-(methylethylidene) moiety (FIG. 1) of diacetonefructose that in turn have desirable specificity for topiramate without cross-reactivity to, for example, 9-hydroxytopiramate, a metabolite of topiramate. The antibodies may be used in methods for detecting topiramate in a sample. Signal generating conjugates are prepared and may be employed in the above methods.

In an exemplary embodiment, the diacetonefructose shown in FIG. 1 is the starting material for the synthesis of haptens and immunogens. The unique chemical structure of diacetonefructose is useful in the development of a specific assay for topiramate. That is, 2,3:4,5-bis-O-(methylethylidene) moiety is a common epitope of both topiramate and diacetonefructose, but diacetonefructose is void of the sulfamate group present in topiramate. The shared specific chemical structure of 2,3:4,5-bis-O-(methylethylidene) is retained to prepare immunogens and raise antibodies accordingly.

In one embodiment, the present invention provides for the design of diacetonefructose haptens and immunogens by conversion of the hydroxyl group to a primary amine (10). Examples of diacetonefructose haptens (10, 12, 19, 22, and 25) and immunogens (10-KLH, 12-KLH, 12-L-KLH, 19-KLH, 22-KLH and 25-KLH) are shown in the Examples. Placement of a linker on the modified hydroxyl group of diacetonefructose for preparation of immunogens yields a generation of antibodies that detect both diacetonefructose and topiramate because they share the specificity of the 2,3:4,5-bis-O-(methylethylidene) epitope. Antibodies that are specific to diacetonefructose derivatives may be utilized for different immunoassay formats. The present invention thus provides a series of diacetonefructose derivatives and immunogens useful within various types of immunoassays.

### Operative Groups

### Immunogenic Carriers

The compounds of the invention such as diacetonefructose derivatives can be made immunogenic by coupling them to a suitable immunogenic carrier. An immunogenic carrier is a group which, when conjugated to a hapten and injected into a mammal, will induce an immune response and elicit the production of antibodies. Immunogenic carriers are also referred to as antigenic carriers and by other synonyms common in the art.

The molecular weight of immunogenic carriers typically range from about 2,000 to 10⁷, usually from about 20,060 to 600,000, and more usually from about 25,000 to 250,000 molecular weight. In one embodiment, there is as least about one per 150,000 molecular weight, at least one group per 50,000 molecular weight, or at least one group per 25,000 molecular weight.

Various protein types may be employed as the immunogenic carrier. These types include albumins, serum proteins, e.g., globulins, ocular lens proteins, lipoproteins, etc. Illustrative proteins include bovine serum albumin (BSA), human serum albumin (HSA), keyhole limpet hemocyanin (KLH), egg ovalbumin, bovine gamma-globulin (BGG), etc. Alternatively, synthetic polypeptides may be utilized.

The immunogenic carrier can also be a polysaccharide, which is a high molecular weight polymer built up by repeated condensations of monosaccharides. Examples of polysaccharides are starches, glycogen, cellulose, carbohydrate gums, such as gum arabic, agar, and so forth. The polysaccharide can also contain peptides and/or lipid residues.

The immunogenic carrier can also be a poly(nucleic acid) either alone or conjugated to one of the above mentioned polypeptides or polysaccharides.

The immunogenic carrier can also be a particle. The particles can be at least about 0.02 microns and not more than about 100 microns, at least about 0.05 microns and less than about 20 microns, or from about 0.3 to about 10 microns in diameter. The particle may be organic or inorganic, swellable or non-swellable, porous or non-porous, preferably of a density approximating water, generally from about 0.7 to 1.5 g/mL, and composed of material that can be transparent, partially transparent, or opaque. The particles can be biological materials such as cells and microorganisms, e.g., erythrocytes, leukocytes, lymphocytes, hybridomas, Streptococcus, Staphylococcus aureus, Escherichia coli, viruses, and the like. The particles can also comprise organic and inorganic polymers, liposomes, latex particles, phospholipid vesicles, chylomicrons, lipoproteins, and the like.

The polymers can be either addition or condensation polymers. Particles derived therefrom will be readily dispersible in an aqueous medium and may be adsorptive or functionalizable.

The particles can be derived from naturally occurring materials, naturally occurring materials which are synthetically modified, and synthetic materials. Among organic polymers of particular interest are polysaccharides, particularly cross-linked polysaccharides, such agarose, which is available as Sepharose, dextran, available as Sephadex and Sephacryl, cellulose, starch, and the like; addition polymers, such as polystyrene, polyvinyl alcohol, homopolymers and copolymers of derivatives of acrylate and methacrylate, particularly esters and amides having free hydroxyl functionalities, and the like.

The particles will usually be polyfunctional and will be bound to or be capable of binding (being conjugated) to a diacetonefructose derivative.

### Signal Generating Moiety

In the methods and compositions of this invention, a variety of signal-generating moieties can be employed. These moieties may include radioactive isotopes, synthetic polymers, saccharides (including monosaccharides and polysaccharides), peptides (including single amino acids and poly(amino acids), i.e., polypeptides), proteins, enzymes, enzyme fragments, enzyme substrates, enzyme inhibitors, coenzymes, catalysts, fluorophores, dyes, chemiluminescers, luminescers, sensitizers, non-magnetic or magnetic particles, solid supports, liposomes, ligands, receptors, hapten radioactive isotopes, and the like. Signal generating moieties appropriate for the invention can also be found in textbooks or catalogs, such as Handbook of Fluorescent Probes and Research Products, 9^{th} ed., Richard Haugland, ed. (Molecular Probes, 2003), which is herein incorporated by reference. Chapter 7 of the Handbook is especially useful for selecting signal generating moieties that are appropriate for use in the invention.

Signal generating moieties may be attached to a compound described herein, such as a diacetonefructose derivative, directly or through a linker, and may also be attached to receptors of the invention, as described elsewhere herein. The signal generating moieties discussed herein can be utilized in the immunoassays and kits of the invention.

### Fluorophores

For the purposes of the invention a fluorophore can be a substance which itself fluoresces, can be made to fluoresce, or can be a fluorescent analogue of an analyte.

In principle, any fluorophore can be used in the assays of this invention. Useful fluorophores, however, have the following characteristics:
a. A fluorescence lifetime of greater than about 15 ns;
b. An excitation wavelength of greater than about 350 nm;
c. A Stokes shift (a shift to lower wave-length of the emission relative to absorption) of greater than about 20 nm;
d. For homogeneous assays, fluorescence lifetime should vary with binding status; and
e. The absorptivity and quantum yield of the fluorophore should be high.

The longer lifetime is advantageous because it is easier to measure and more easily distinguishable from the Raleigh scattering (background). Excitation wavelengths greater than 350 nm reduce background interference because most fluorescent substances responsible for background fluorescence in biological samples are excited below 350 nm. A greater Stokes shift also allows for less background interference.

The fluorophore should have a functional group available for conjugation either directly or indirectly to the diacetonefructose derivative or receptor. An additional criterion in selecting the fluorophore is the stability of the fluorophore: it should not be photophysically unstable, and it should be relatively insensitive to the assay conditions, e.g., pH, polarity, temperature and ionic strength.

In an exemplary embodiment, fluorophores for use in heterogeneous assays are relatively insensitive to binding status. In contrast, in one embodiment, fluorophores for use in homogeneous assays must be sensitive to binding status, i.e., the fluorescence lifetime must be alterable by binding so that bound and free forms can be distinguished.

Examples of fluorophores useful in the invention are naphthalene derivatives (e.g. dansyl chloride), anthracene derivatives (e.g. N-hydroxysuccinimide ester of anthracene propionate), pyrene derivatives (e.g. N-hydroxysuccinimide ester of pyrene butyrate), fluorescein derivatives (e.g. fluorescein isothiocyanate), rhodamine derivatives (e.g. rhodamine isothiocyanate), phycoerythin, and Texas Red.

### Enzymes

In an exemplary embodiment, the signal generating moiety is an enzyme. From the standpoint of operability, a very wide variety of enzymes can be used. Useful enzymes include those that are stable when stored for a period of at least three months, and preferably at least six months at temperatures which are convenient to store in the laboratory, normally −20° C. or above. Useful enzymes may also have a satisfactory turnover rate at or near the pH optimum for binding to the receptor, which is normally at about pH 6-10, usually 6.0 to 8.0. In one embodiment, a product is formed or destroyed as a result of the enzyme reaction which absorbs light in the ultraviolet region or the visible region, that is, the range of about 250-750 nm or 300-600 nm. The enzyme may also have a substrate (including cofactors) which has a molecular weight in excess of 300 or in excess of 500, there being no upper limit. In one embodiment, there is no naturally occurring inhibitors for the enzyme present in fluids to be assayed.

In one embodiment, enzymes of up to 600,000 molecular weight can be employed. In an exemplary embodiment, relatively low molecular weight enzymes will be employed of from about 10,000 to about 300,000 molecular weight, from about 10,000 to about 150,000 molecular weight, and from about 10,000 to about 100,000 molecular weight. Where an enzyme has a plurality of subunits the molecular weight limitations refer to the enzyme and not to the subunits.

Enzymes can be useful in the invention include alkaline phosphatase, horseradish peroxidase, lysozyme, glucose-6-phosphate dehydrogenase, lactate dehydrogenase, β-galactosidase, and urease. Also, a genetically engineered fragment of an enzyme may be used, such as the donor and acceptor fragment of β-galactosidase utilized in CEDIA immunoassays (see Henderson DR et al., Clin Chem. 32(9):1637-1641 (1986)); U.S. Pat. No. 4,708,929. These and other enzymes which can be used have been discussed in detail by Eva Engvall in Enzyme Immunoassay ELISA and EMIT in Methods in Enzymology, 70:419-439 (1980) and in U.S. Pat. No. 4,857,453.

Enzymes, enzyme fragments, enzyme inhibitors, enzyme substrates, and other components of enzyme reaction systems can be attached to the haptens and receptors, and employed in the immunoassays of the invention. Where any of these components is used as a signal generating moiety, a chemical reaction involving one of the components is part of the signal producing system.

Coupled catalysts can also involve an enzyme with a non-enzymatic catalyst. The enzyme can produce a reactant, which undergoes a reaction catalyzed by the non-enzymatic catalyst or the non-enzymatic catalyst may produce a substrate (includes coenzymes) for the enzyme. A wide variety of non-enzymatic catalysts, which may be employed are found in U.S. Pat. No. 4,160,645, which is incorporated herein by reference in its entirety.

The enzyme or coenzyme employed may provide the desired amplification by producing a product that absorbs light, e.g., a dye, or emits light upon irradiation, e.g., a fluorescer. Alternatively, the catalytic reaction can lead to direct light emission, e.g., chemiluminescence. A large number of enzymes and coenzymes for providing such products are indicated in U.S. Pat. No. 4,275,149, columns 19 to 23, and U.S. Pat. No. 4,318,980, columns 10 to 14. The entire disclosures of U.S. Pat. No. 4,275,149 and U.S. Pat. No. 4,318,980 are incorporated by reference.

A number of enzyme combinations are set forth in U.S. Pat. No. 4,275,149, columns 23 to 28, which combinations can find use in the subject invention. The entire disclosure of U.S. Pat. No. 4,275,149 is incorporated by reference

When a single enzyme is used as a label, such enzymes that may find use are hydrolases, transferases, lyases, isomerases, ligases or synthetases and oxidoreductases. In an exemplary embodiment, the enzyme is a hydrolase. In one embodiment, luciferases may be used such as firefly luciferase and bacterial luciferase. Illustrative dehydrogenases include malate dehydrogenase, glucose-6-phosphate dehydrogenase, and lactate dehydrogenase. Illustrative oxidases include glucose oxidase. Of the peroxidases, horse radish peroxidase is illustrative. Of the hydrolases, alkaline phosphatase, β-glucosidase and lysozyme are illustrative.

Of interest are enzymes which involve the production of hydrogen peroxide and the use of the hydrogen peroxide to oxidize a dye precursor to a dye. Exemplary combinations include saccharide oxidases, e.g., glucose and galactose oxidase, or heterocyclic oxidases, such as uricase and xanthine oxidase, coupled with an enzyme which employs the hydrogen peroxide to oxidize a dye precursor, that is, a peroxidase such as horse radish peroxidase, lactoperoxidase, or microperoxidase. Additional enzyme combinations may be found in the subject matter incorporated by reference.

Enzymes that employ nicotinamide adenine dinucleotide (NAD) or its phosphate (NADP) as a cofactor can be used. Exemplary enzymes include glucose-6-phosphate dehydrogenase and NAD-dependent glucose-6-phosphate dehydrogenase.

### Methods of Attaching a Hapten to an Operative Group

There are many options available for the conjugation of a hapten with a operative group. In an exemplary embodiment, the hapten comprises a reactive functional group, and is conjugated to the operative group. In another exemplary embodiment, the operative group is activated, and then conjugated to the hapten.

The methods of attaching are dependent upon the reactive functional groups present at the site of activation. In an exemplary embodiment, the reactive functional group of the haptens of the invention and the reactive functional group of an operative group comprise electrophiles and nucleophiles that can generate a covalent linkage between them. Alternatively, the reactive functional group comprises a photoactivatable group, which becomes chemically reactive only after illumination with light of an appropriate wavelength. Typically, the conjugation reaction between the reactive functional group and the operative group results in one or more atoms of the reactive functional group or the operative group being incorporated into a new linkage attaching the hapten to the operative group. Selected examples of functional groups and linkages are shown in Table 1, where the reaction of an electrophilic group and a nucleophilic group yields a covalent linkage.

TABLE 1
Examples of some routes to useful covalent linkages with electrophile
and nucleophile reactive groups

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

*Activated esters, as understood in the art, generally have the formula —COΩ, where Ω is a good leaving group (e.g. oxysuccinimidyl (—OC₄H₄O₂) oxysulfosuccinimidyl (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates

Where the reactive functional group is an activated ester of a carboxylic acid, such as a succinimidyl ester of a carboxylic acid, the resulting compound is useful for preparing conjugates of carrier molecules such as proteins, nucleotides, oligonucleotides, or haptens. Where the reactive group is a maleimide or haloacetamide the resulting compound is particularly useful for conjugation to thiol-containing substances. Where the reactive group is a hydrazide, the resulting compound is particularly useful for conjugation to periodate-oxidized carbohydrates and glycoproteins, and in addition is an aldehyde-fixable polar tracer for cell microinjection. Where the reactive group is a silyl halide, the resulting compound is particularly useful for conjugation to silica surfaces, particularly where the silica surface is incorporated into a fiber optic probe subsequently used for remote ion detection or quantitation.

Conjugation of haptens typically involves first dissolving the hapten in water or a water-miscible such as a lower alcohol, dimethylformamide (DMF), dimethylsulfoxide (DMSO), acetone, acetonitrile, tetrahydrofuran (THF), dioxane or acetonitrile. These methods are been described in detail in Hermanson Greg T., Bioconiugate Techniques, Chapter 9, p. 419-455, Academic Press, Inc., 1996. Conjugates typically result from mixing appropriate reactive compounds and the component to be conjugated in a suitable solvent in which both are soluble, using methods well known in the art, followed by separation of the conjugate from any unreacted component and by-products. These present compounds are typically combined with the component under conditions of concentration, stoichiometry, pH, temperature and other factors that affect chemical reactions that are determined by both the reactive groups on the compound and the expected site of modification on the component to be modified. These factors are generally well known in the art of forming bioconjugates (Haugland et al., “Coupling of Antibodies with Biotin”, The Protein Protocols Handbook, J. M. Walker, ed., Humana Press, (1996); Haugland “Coupling of Monoclonal Antibodies with Fluorophores”, Methods in Molecular Biology, Vol. 45: Monoclonal Antibody Protocols, W. C. Davis, ed. (1995)). For those reactive compounds that are photoactivated, conjugation requires illumination of the reaction mixture to activate the reactive compound. The labeled component is used in solution or lyophilized and stored for later use.

### Receptors

Included within the invention are receptors specific for topiramate and/or diacetonefructose derivatives described herein. In an exemplary embodiment, the receptor is an antibody. In another exemplary embodiment, the receptor comprises the antigen-binding residues of an antibody. In another exemplary embodiment, the receptor can further comprise a signal generating moiety as discussed herein. The methods of attaching the signal generating moieties to the compounds of the invention such as diacetonefructose derivatives are applicable to the methods of attaching the signal generating moieties to the receptors of the invention.

### Antibodies

Antibodies, or immunoglobulins, are molecules produced by organs of the immune system to defend against antigens. The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Cellular and Molecular Immunology Ch. 3 (Abbas and Lichtman, ed., 5th ed. Saunders (2003)) (incorporated by reference in its entirety for all purposes). The variable regions of each light/heavy chain pair form the antibody binding site. Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

Antibodies exist as intact immunoglobulins or as a number of well-characterized fragments. Basic antibody fragments include Fab, which consists of portions of a heavy chain (above the hinge region) and a light chain, and Fab′, which is essentially Fab with part of the hinge region attached. Peptidases digest the antibody in different ways to produce fragments with combinations of these basic antibody fragments. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments.

Antibodies specific for the antigens of the invention may be produced by in vitro or in vivo techniques. In vitro techniques may involve exposure of lymphocytes to the diacetonefructose derivatives, while in vivo techniques, such as the production of polyclonal and monoclonal antibodies, require the injection of diacetonefructose derivative antigens into a suitable vertebrate host.

In one aspect, the invention provides methods of making an antibody comprising administering to a subject a compound of the invention. In an exemplary embodiment, the subject is an animal or a cell.

In one embodiment, a diacetonefructose derivative-based immunogen in accordance with the present invention can be used for producing monoclonal and/or polyclonal antibodies. As such, antibodies can be produced from the diacetonefructose derivative-based immunogen and interact and/or bind with topiramate. This can allow for the derivatives of the present invention to be useful in preparing specific antibodies for use in immunoassays for identifying the presence of topiramate.

In one aspect, the invention provides complexes comprising an antibody and a compound described herein. In one embodiment, the antibody specifically binds to a compound described herein. In one embodiment, the antibody specifically binds to 2,3:4,5-bis-O-methylethylidene. In one embodiment, the complex comprises an antibody and a compound of the invention. In an exemplary embodiment, the complex comprises an antibody and a diacetonefructose derivative. In an exemplary embodiment, the complex comprises an antibody and a diacetonefructose derivative conjugate. In an exemplary embodiment, the complex comprises the antibody and topiramate. In an exemplary embodiment, the antibody of the above mentioned complexes is raised against a diacetonefructose derivative as described herein.

In one aspect, the invention provides an antibody that binds topiramate and has less than about 10% cross-reactivity with a member selected from a drug and a metabolite of topiramate, wherein the drug is not topiramate. In an exemplary embodiment, the antibody binds topiramate and has less than about 5% or less than about 3% or less than about 1% cross-reactivity with a member selected from a drug and a metabolite of topiramate, wherein the drug is not topiramate. In one embodiment, the drug is a member selected from an antiepileptic drug, an antiinflammatory drug, an anticonvulsive drug, and an antibacterial sulfonamide, wherein the drug is not topiramate. In an exemplary embodiment, the antiepileptic drug is phenytoin, the antiinflammatory drug is ibuprofen and the anticonvulsive drug is tiagabine. In one embodiment, the metabolite is a member selected from 9-hydroxytopiramate, 10-hydroxytopiramate, 2,3-diol-topiramate and 4,5-diol-topiramate.

The following method may be employed to prepare polyclonal antibodies using any of the immunogenic compounds described herein. In an exemplary embodiment, the immunogenic compound is a diacetonefructose derivative conjugate described herein. Antiserum containing antibodies is obtained by well-established techniques involving immunization of an animal, such as rabbits and sheep, with an appropriate immunogen and obtaining antisera from the blood of the immunized animal after an appropriate waiting period. State-of-the-art reviews are provided by Parker, Radioimmunoassay of Biologically Active Compounds, Prentice-Hall (Englewood Cliffs, N.J., U.S., 1976), Butler, J. Immunol. Meth. 7: 1 24 (1975); Broughton and Strong, Clin. Chem. 22: 726 732 (1976); and Playfair, et al., Br. Med. Bull. 30: 24 31 (1974).

The following procedure may be employed to prepare monoclonal antibodies, in particular for the immunogenic compounds described herein. In an exemplary embodiment, the immunogenic compound is a diacetonefructose derivative conjugate described herein. Monoclonal antibodies were produced according to the standard techniques of Kohler and Milstein, Nature 265:495 497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3 46 (1981). Samples of an appropriate immunogen preparation are injected into an animal such as a mouse and, after a sufficient time, the animal is sacrificed and spleen cells obtained. Alternatively, the spleen cells of an non-immunized animal can be sensitized to the immunogen in vitro. The spleen cell chromosomes encoding the base sequences for the desired immunoglobulins can be compressed by fusing the spleen cells, generally in the presence of a non-ionic detergent, for example, polyethylene glycol, with a myeloma cell line. The resulting cells, which include fused hybridomas, are allowed to grow in a selective medium, such as HAT-medium, and the surviving immortalized cells are grown in such medium using limiting dilution conditions. The cells are grown in a suitable container, e.g., microtiter wells, and the supernatant is screened for monoclonal antibodies having the desired specificity.

Various techniques exist for enhancing yields of monoclonal antibodies, such as injection of the hybridoma cells into the peritoneal cavity of a mammalian host, which accepts the cells, and harvesting the ascites fluid. Where an insufficient amount of the monoclonal antibody collects in the ascites fluid, the antibody is harvested from the blood of the host. Alternatively, the cell producing the desired antibody can be grown in a hollow fiber cell culture device or a spinner flask device, both of which are well known in the art. Various conventional ways exist for isolation and purification of the monoclonal antibodies from other proteins and other contaminants (see Kohler and Milstein, supra).

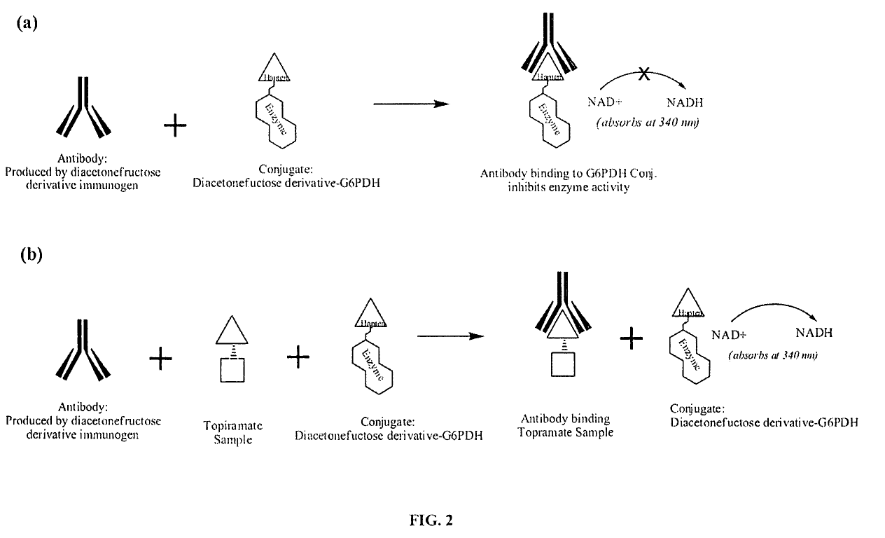

In general, antibodies can be purified by known techniques such as chromatography, e.g., DEAE chromatography, ABx chromatography, and the like, filtration, and so forth. Antibodies may be screened using any of several techniques, for example using a homogeneous enzyme immunoassay format as illustrated in FIG. 2, and considering such properties as specificity, enzyme conjugate inhibition, calibration curve size and specificity.

Cross-reactivity testing is performed by adding known amounts of cross reactant into human serum. The instrument used for this evaluation is the Roche Cobas Mira Chemistry Analyzer. A homogeneous enzyme immunoassay technique which can be used for the analysis is based on competition between a drug in the sample and drug labeled with the enzyme glucose-6-phosphate dehydrogenase (G6PDH) for receptor binding sites. Enzyme activity decreases upon binding to the antibody, so the drug concentration in the sample can be measured in terms of enzyme activity. Active enzyme converts nicotinamide adenine dinucleotide (NAD) to NADH, resulting in an absorbance change that is measured spectrophotometrically. Endogenous serum G6PDH does not interfere because the coenzyme functions only with the bacterial (Leuconostoc mesenteroides) enzyme employed in the assay. The quantitative analysis of drugs can be performed using human urine, serum, plasma, whole blood, or ultra filtrate.

### Other Receptors

Receptors can comprise the antigen-binding domains or amino acids critical for antigen binding, e.g. antigen-binding residues, of an antibody that specifically binds topiramate or diacetonefructose derivative. Such antigen-binding domains or residues can comprise the Complementarity-Determining Region (CDR) of an antibody. The receptors can also structurally mimic the structure represented by the antigen-binding domains or residues of a CDR. For example, if there are four amino acids within the CDR of an antibody that are critical for binding the antigen to the antibody, e.g. antigen-binding residues, then a receptor of the invention need only possess those four critical amino acids structurally arranged so as to substantially mimic their structural arrangement within the CDR of the antibody. The linkages between the critical amino acids are only important to the extent that they structurally mimic the CDR of the antibody. In this example, substitution of isosteres of the critical amino acids, such as aspartic acid for glutamic acid, are allowed.

### Immunoassays

Quantitative, semiquantitative, and qualitative methods as well as all other methods for determining topiramate are considered to be methods of measuring the amount of topiramate. For example, a method which merely detects the presence or absence of topiramate in a sample suspected of containing an topiramate is considered to be included within the scope of the present invention.

Synonyms for the phrase “measuring the amount of topiramate” which are contemplated within the scope of the present invention include, but are not limited to, detecting, measuring, or determining topiramate; detecting, measuring, or determining the presence of topiramate; and detecting, or determining the amount of topiramate. In one embodiment, measuring the amount of topiramate occurs by measuring the amount of a topiramate complex or a topiramate conjugate. In one embodiment, a topiramate complex or a topiramate conjugate comprises an antibody. Measuring the amount of topiramate can occur either by directly detecting a topiramate complex or topiramate conjugate or indirectly detecting the topiramate complex or topiramate conjugate.

In the TDM field there are several categories of methods available for determining the presence or the concentration of topiramate in a sample. One such category is immunoassays, which are currently used to determine the presence or concentration of various analytes in biological samples, both conveniently and reliably (The Immunoassay Handbook, edited by David Wild, M Stockton Press, 1994). Generally speaking, immunoassays utilize specific receptors to target analytes in fluids, where at least one such receptor is generally labeled with one of a variety of signal-generating moieties.

Immunoassays usually are classified in one of several ways. One method is according to the mode of detection used, i.e., enzyme immunoassays, radio immunoassays, fluorescence polarization immunoassays, chemiluminescence immunoassays, turbidimetric assays, etc. Another grouping method is according to the assay procedure used, i.e., competitive assay formats, sandwich-type assay formats as well as assays based on precipitation or agglutination principles. In one application, a further distinction is made depending on whether washing steps are included in the procedure (so-called heterogeneous assays) or whether reaction and detection are performed without a washing step (so-called homogeneous assays). All the essential terms, procedures and devices are known to the skilled artisan from text books in the field, e.g., “Manual of Immunological Methods”, eds. P. Brousseau and M. Beaudet, CRC Press, 1998, and “Practice and Theory of Enzyme Immunoassays”, eds. P. Tijssen and R. H. Burdon, Elsevier Health Sciences, 1985, are herewith included by reference.

### Homogeneous and Heterogeneous Immunoassays

As mentioned above, immunoassays may be heterogeneous or homogeneous. Heterogeneous immunoassays have been applied to both small and large molecular weight analytes and require separation of bound materials (to be detected or determined) from free materials (which may interfere with that determination). Heterogeneous immunoassays may comprise a receptor or an antigen immobilized on solid surfaces such as plastic microtiter plates, beads, tubes, or the like or on membrane sheets, chips and pieces of glass, nylon, cellulose or the like (“Immobilized Enzymes, Antigens, Antibodies, and Peptides”, ed. Howard H. Weetall, Marcel Dekker, Inc., 1975). In heterogeneous immunoassays, antigen-receptor (e.g., antibody) complexes bound to the solid phase are separated from unreacted and non-specific analyte in solution, generally by centrifugation, filtration, precipitation, magnetic separation or aspiration of fluids from solid phases, followed by repeated washing of the solid phase-bound antigen-receptor complex. The solid phase-bound complex is subsequently involved in a detection step.

Homogeneous assays are, in general, liquid phase procedures that do not utilize antigens or receptors (e.g., antibodies) that are immobilized on solid materials. Separation and washing steps are not required. In an exemplary embodiment, the antigens or receptors comprise a fluorophore signal-generating moiety, which upon forming a complex of the antigen and receptor binding pair competitively with a target analyte undergoes an excitation or quenching of fluorescence emission. In an exemplary embodiment, the antigens or receptors comprise an enzyme signal-generating moiety, which upon forming a complex of the antigen and receptor binding pair competitively with a target analyte undergoes an enhancement or a reduction in enzyme product formation, due to a conformational change in the enzyme upon forming the complex of the binding pair. Homogeneous methods have typically been developed for the detection of haptens and small molecules, such as drugs, hormones and peptides.

Thus, in one aspect, the invention provides methods of determining an amount of topiramate in a sample comprising (a) contacting the sample with an antibody raised against a compound of the invention, thus yielding an antibody-topiramate complex; and (b) detecting the antibody-topiramate complex. In an exemplary embodiment, the compound is a diacetonefructose derivative conjugate. In one embodiment, the method further comprises contacting the sample with a ligand competitively binding to the antibody. In one embodiment, the ligand is a compound of the invention. In an exemplary embodiment, the ligand is a diacetonefructose derivative. In an exemplary embodiment, the ligand is a diacetonefructose derivative conjugate. In one embodiment, the antibody has less than about 10% cross-reactivity with a member selected from a drug and a metabolite of topiramate, wherein the drug is not topiramate. In one embodiment, the drug is a member selected from an antiepileptic drug, an antiinflammatory drug, an anticonvulsive drug, and an antibacterial sulfonamide, wherein the drug is not topiramate. In an exemplary embodiment, the antiepileptic drug is phenytoin, the antiinflammatory drug is ibuprofen and the anticonvulsive drug is tiagabine. In one embodiment, the metabolite is a member selected from 9-hydroxytopiramate, 10-hydroxytopiramate, 2,3-diol-topiramate and 4,5-diol-topiramate. In one embodiment the antibody has about 9%, about 8%, about 7%, about 6%, about 5%, about 4%, about 3%, about 2% or about 1% cross-reactivity with a drug and/or a metabolite of topiramate, wherein the drug is not topiramate.

### Detection

### Fluorescence

When a fluorescently labeled analyte, such as for example topiramate, diacetonefructose derivative conjugate, or antibody-diacetonefructose derivative conjugate complex is employed, the fluorescence emitted is proportional (either directly or inversely) to the amount of analyte. The amount of fluorescence is determined by the amplitude of the fluorescence decay curve for the fluorescent species. This amplitude parameter is directly proportional to the amount of fluorescent species and accordingly to the analyte, such as for example topiramate, diacetonefructose derivative conjugate, or antibody-diacetonefructose derivative conjugate complex.

In general spectroscopic measurement of fluorescence is accomplished by:
a. exciting the fluorophore with a pulse of light;
b. detecting and storing an image of the excitation pulse and an image of all the fluorescence (the fluorescent transient) induced by the excitation pulse;
c. digitizing the image;
d. calculating the true fluorescent transient from the digitized data;
e. determining the amplitude of the fluorescent transient as an indication of the amount of fluorescent species.

According to the method, substantially all of the fluorescence emitted by the fluorescent species reaching the detector as a function of time from the instant of excitation is measured. As a consequence, the signal being detected is a superimposition of several component signals (for example, background and one analyte specific signal). As mentioned, the individual contributions to the overall fluorescence reaching the detector are distinguished based on the different fluorescence decay rates (lifetimes) of signal components. In order to quantify the magnitude of each contribution, the detected signal data is processed to obtain the amplitude of each component. The amplitude of each component signal is proportional to the concentration of the fluorescent species.

### Enzymes

Detection of the amount of product produced by the diacetonefructose derivative conjugate comprising, for example, an enzyme, can be accomplished by several methods which are known to those of skill in the art. Among these methods are colorimetry, fluorescence, and spectrophotometry. These methods of detection are discussed in “Analytical Biochemistry” by David Holme, Addison-Wesley, 1998, which is incorporated herein by reference.

In one embodiment, a change in activity of an enzyme is sufficient to allow detection of a diacetonefructose derivative conjugate when the enzyme is used as a label. In one embodiment, the enzyme's activity is reduced from about 10% to about 100%, from about 20% to about 99%, or from about 30% to about 95%, from about 40% to about 95%, from about 50% to about 95%, from about 10% to about 50%, from about 10% to about 40%, and from about 10% to about 30%.

### Lateral Flow Chromatography

The compounds and methods of the invention also encompass the use of these materials in lateral flow chromatography technologies. The essence of lateral flow chromatography involves a membrane strip which comprises a detection device, such as a signal generating moiety. A sample from a patient is then applied to the membrane strip. The sample interacts with the detection device, producing a result. The results can signify several things, including the absence, presence or concentration of an analyte, such as for example topiramate, diacetonefructose derivative conjugate, or antibody-diacetonefructose derivative conjugate complex in the sample.

In one embodiment, the invention provides a method of qualitatively determining the presence or absence of topiramate in a sample, through the use of lateral flow chromatography. The basic design of the qualitative lateral flow device is as follows: 1) The sample pad is where the sample is applied. The sample pad is treated with chemicals such as buffers or salts, which, when redissolved, optimize the chemistry of the sample for reaction with the conjugate, test, and control reagents. 2) Conjugate release pad is typically a polyester or glass fiber material that is treated with a conjugate reagent such as an antibody colloidal gold conjugate. A typical process for treating a conjugate pad is to use impregnation followed by drying. In use, the liquid sample added to the test will redissolve the conjugate so that it will flow into the membrane. 3) The membrane substrate is usually made of nitrocellulose or a similar material whereby antibody capture components are immobilized. 4) A wicking pad is used in tests where blood plasma must be separated from whole blood. An impregnation process is usually used to treat this pad with reagents intended to condition the sample and promote cell separation. 5) The absorbent pad acts as a reservoir for collecting fluids that have flowed through the device. 6) The above layers and membrane system are laminated onto a plastic backing with adhesive material which serves as a structural member.

In one embodiment, the invention provides a method of qualitatively determining the presence of topiramate in a sample, through the use of lateral flow chromatography. In this embodiment, the membrane strip comprises a sample pad, which is a conjugate release pad (CRP), which comprises a receptor that is specific for topiramate. In an exemplary embodiment, the receptor is as described herein. In an exemplary embodiment, the receptor is raised against a compound of the invention. This receptor is conjugated to a signal-generating moiety, such as a colloidal gold particle. Other detection moieties useful in a lateral flow chromatography environment include dyes, colored latex particles, fluorescently labeled latex particles, non-isotopic signal generating moieties, etc. The membrane strip further comprises a capture line, in which topiramate is immobilized on the strip. In some embodiments, this immobilization is through covalent attachment to the membrane strip, optionally through a linker. In other embodiments, the immobilization is through non-covalent attachment to the membrane strip. In still other embodiments, the topiramate in the capture line is attached to a reactive partner, such as an immunogenic carrier like BSA.

Sample from a patient is applied to the sample pad, where it can combine with the receptor in the CRP, thus forming a solution. This solution is then allowed to migrate chromatographically by capillary action across the membrane. When the topiramate is present in the sample, a topiramate-receptor complex is formed, which migrates across the membrane by capillary action. When the solution reaches the capture line, the topiramate-receptor complex will compete with the immobile topiramate for the limited binding sites of the receptor. When a sufficient concentration of topiramate is present in the sample, it will fill the limited receptor binding sites. This will prevent the formation of a colored receptor-immobile topiramate complex in the capture line. Therefore, absence of color in the capture line indicates the presence of topiramate in the sample.

In the absence of topiramate in the sample, a colored receptor-immobile topiramate complex will form once the solution reaches the capture line of the membrane strip. The formation of this complex in the capture line is evidence of the absence of topiramate in the sample.

In one embodiment, the invention provides a method of quantitatively determining the amount of topiramate in a sample through the use of lateral flow chromatography. This technology is further described in U.S. Pat. Nos. 4,391,904; 4,435,504; 4,959,324; 5,264,180; 5,340,539; and 5,416,000, among others, which are herein incorporated by reference. In one embodiment, the receptor is immobilized along the entire length of the membrane strip. In general, if the membrane strip is made from paper, the receptor is covalently bound to the membrane strip. If the membrane strip is made from nitrocellulose, then the receptor can be non-covalently attached to the membrane strip through, for example, hydrophobic and electrostatic interactions.

The membrane strip comprises a CRP which comprises topiramate attached to a detector moiety. In an exemplary embodiment, the detector moiety is an enzyme, such as horseradish peroxidase (HRP).

Sample from a patient is applied to the membrane strip, where it can combine with the topiramate/detector molecule in the CRP, thus forming a solution. This solution is then allowed to migrate chromatographically by capillary action across the membrane. When topiramate is present in the sample, both the sample topiramate and the topiramate/detector molecule compete for the limited binding sites of the receptor. When a sufficient concentration of topiramate is present in the sample, it will fill the limited receptor binding sites. This will force the topiramate/detector molecule to continue to migrate in the membrane strip. The shorter the distance of migration of the topiramate/detector molecule in the membrane strip, the lower the concentration of topiramate in the sample, and vice versa. When the topiramate/detector molecule comprises an enzyme, the length of migration of the topiramate/detector molecule can be detected by applying an enzyme substrate to the membrane strip. Detection of the product of the enzyme reaction is then utilized to determine the concentration of the topiramate in the sample. In another exemplary embodiment, the enzyme's color producing substrate such as a modified N,N-dimethylaniline is immobilized to the membrane strip and 3-methyl-2-bezothiazolinone hydrazone is passively applied to the membrane, thus alleviating the need for a separate reagent to visualize the color producing reaction.

### Systems for Assaying Topiramate

In one aspect, the invention provides for a signal producing system that is utilized in assays for topiramate. The signal producing system generates a signal that relates to the presence or amount of topiramate in a sample. In one embodiment, the system comprises G6PDH. In one embodiment, the system comprises an antibody raised against a compound of the invention.

In one embodiment, the signal producing system further comprises reagents necessary to produce a measurable signal. Other components of the signal producing system can include substrates, enhancers, activators, chemiluminescent compounds, cofactors, inhibitors, scavengers, metal ions, specific binding substances required for binding of signal generating substances, coenzymes, substances that react with enzymatic products, other enzymes and catalysts, and the like.

The signal producing system provides a signal detectable by external means, normally by measurement of electromagnetic radiation, desirably by visual examination. In an exemplary embodiment, the signal producing system comprises a chromophoric substrate and a G6PDH enzyme of the invention, where chromophoric substrates are enzymatically converted to dyes that absorb light in the ultraviolet or visible region.

Another aspect of the present invention relates to kits useful for conveniently determining the presence or the concentration of topiramate in a sample. In one aspect, the kits of the present invention can comprise a receptor specific for topiramate. In an exemplary embodiment, the receptor is an antibody. In one embodiment, the antibody is raised against a compound of the invention. In one exemplary embodiment, the antibody is raised against a diacetonefructose derivate. In another exemplary embodiment, the receptor comprises the antigen-binding domain or antigen-binding residues that specifically bind to topiramate. In one embodiment, the kits can optionally further comprise calibration and control standards useful in performing the assay; or ancillary reagents. In one embodiment, the kits further comprise instructions for using the kit. The kits can also optionally comprise a diacetonefructose derivative conjugate. To enhance kit versatility, the kit components can be in a liquid reagent form, a lyophilized form, or attached to a solid support. The reagents may each be in separate containers, or various reagents can be combined in one or more containers depending on cross-reactivity and stability of the reagents.

Any sample that is reasonably suspected of containing the analyte topiramate can be analyzed by the kits of the present invention. The sample is typically an aqueous solution such as a body fluid from a host, for example, urine, whole blood, plasma, serum, saliva, semen, stool, sputum, cerebral spinal fluid, tears, mucus, breast milk or the like. In an exemplary embodiment, the sample is plasma or serum. The sample can be pretreated if desired and can be prepared in any convenient medium that does not interfere with the assay. For example, the sample can be provided in a buffered synthetic matrix.

A sample suspected of containing topiramate and a calibration material containing a known concentration of topiramate are assayed under similar conditions. Topiramate concentration may be then calculated by comparing the results obtained for the unknown specimen with results obtained for the standard. This is commonly done by constructing a calibration or dose response curve.

Various ancillary materials will frequently be employed in an assay in accordance with the present invention. For example, buffers will normally be present in the assay medium, as well as stabilizers for the assay medium and the assay components. Frequently, in addition to these additives, additional proteins may be included, such as albumins, or surfactants, particularly non-ionic surfactants, binding enhancers, e.g., polyalkylene glycols, or the like.

In an exemplary embodiment, buffers and/or stabilizers are present in the kit components. In another exemplary embodiment, the kits comprise indicator solutions or indicator “dipsticks”, blotters, culture media, cuvettes, and the like. In yet another exemplary embodiment, the kits comprise indicator cartridges (where a kit component is bound to a solid support) for use in an automated detector. In still another exemplary embodiment, additional proteins, such as albumin, or surfactants, particularly non-ionic surfactants, may be included. In another exemplary embodiment, the kits comprise an instruction manual that teaches a method of the invention and/or describes the use of the components of the kit.

### EXAMPLES

The following examples are offered by way of illustration and not by way of limitation. It should be apparent that the invention can include additional embodiments not illustrated by example. Additionally, many of the examples have been performed with experimental protocols well known in the art using the diacetonefructose derivatives, antigens, immunogens, and anti-diacetonefructose derivative antibodies prepared in accordance with the present invention.

The synthesis of hapten (10) and immunogen (10-KLH) is shown in Examples 1 and 6 respectively. The hydroxyl group of diacetonefructose is converted to a primary amine. The coupling of(10) to KLH (11) is accomplished by activation of the carboxylic group of KLH (11) with 1-ethyl-3-[3-dimethylaminopropyl]-carbodiimide hydrochloride (EDC) followed by reaction with the amine of diacetonefructosefructose derivative (10) to give 10-KLH.

The synthesis of hapten (12, 13), immunogen (12-KLH) and conjugate G6PDH (12-G6PDH) is shown below:

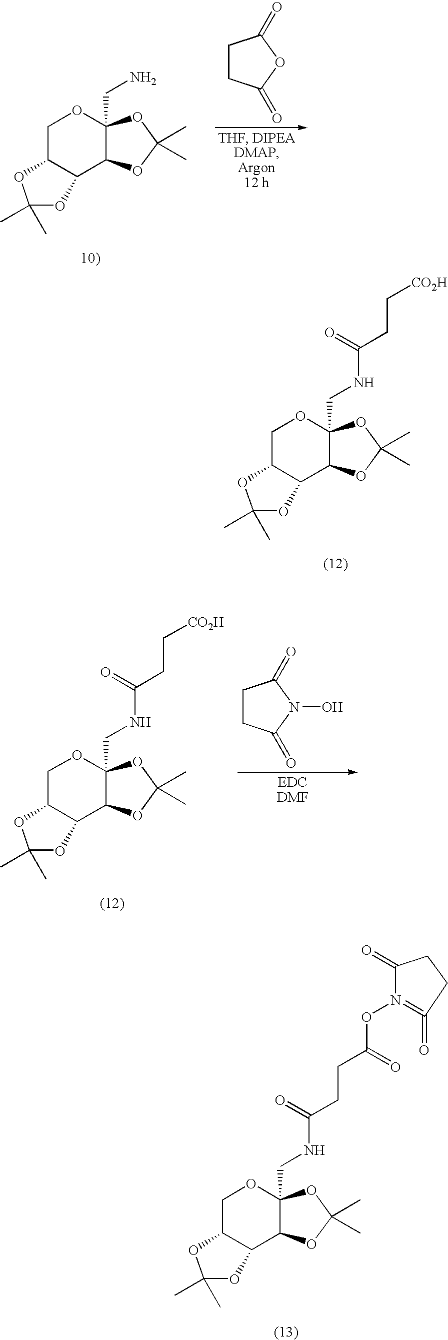

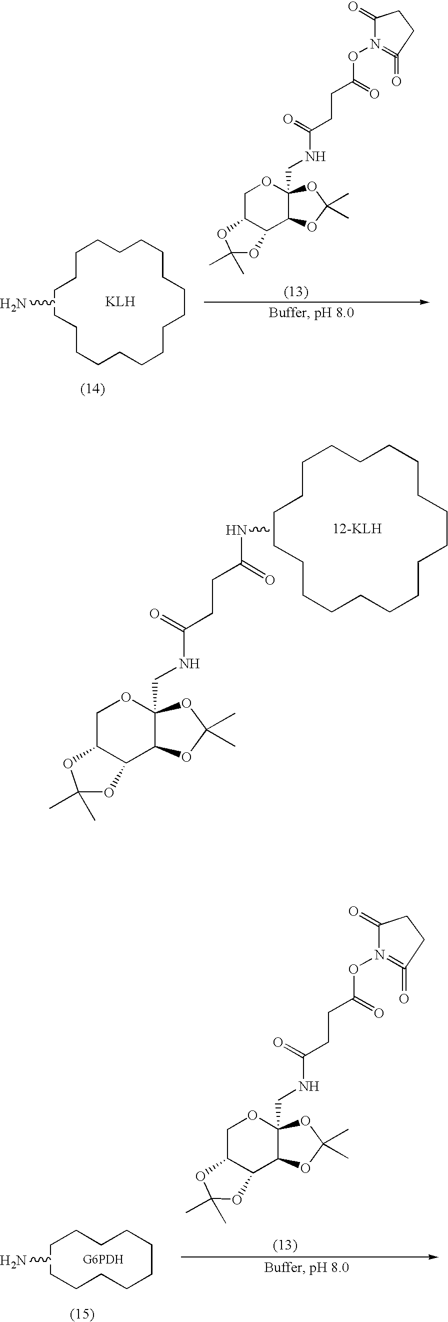

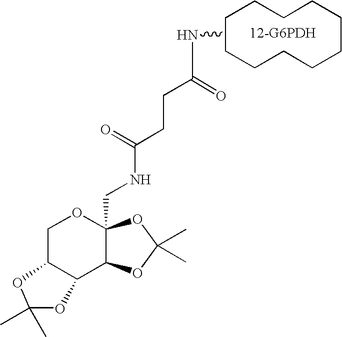

The amine modified hapten (10) was acylated with succinic anhydride to produce hapten 12, which was activated by EDC and NHS to an ester (13), followed by a reaction with amines on KLH (14) and G6PDH (15) to give corresponding immunogen (12-KLH) and conjugate (12-G6PDH) respectively.

Preparation of immunogen (12-L-KLH) is shown in Example 8. KLH (14) was reacted with 6-aminocaproic acid and dimethylamino-propyl-3-ethylcarboiimide hydrochloride to give 6-aminocaproyl KLH (16) (Example 8). 6-aminocaproyl KLH was reacted with hapten (13) in buffer, pH 8.0 to give immunogen (12-L-KLH).

The synthesis of hapten (19, 20), immunogen (19-KLH) and G6PDH conjugate (19- G6PDH) are shown below:

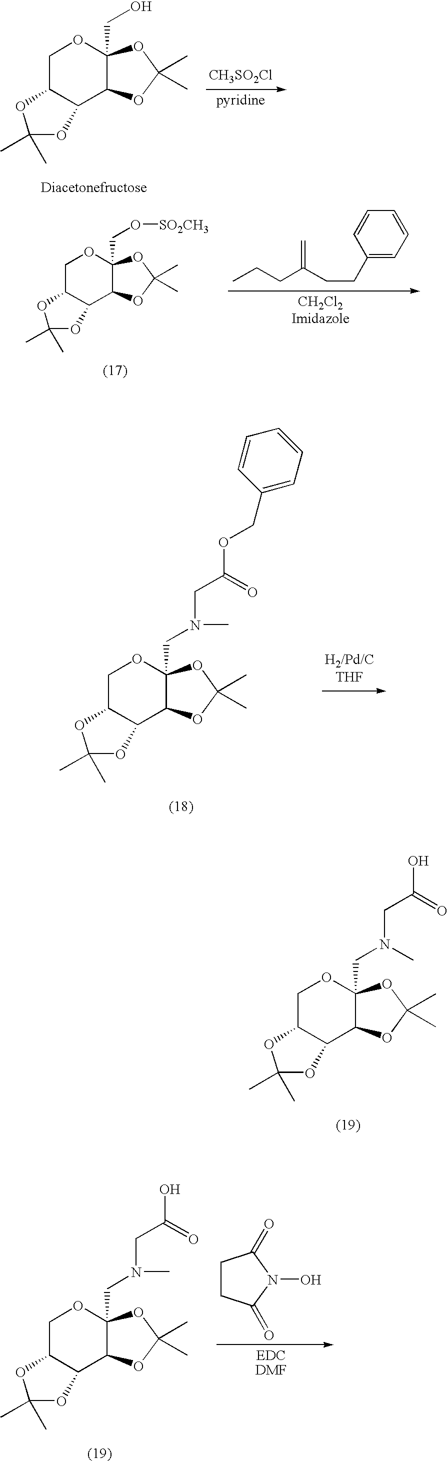

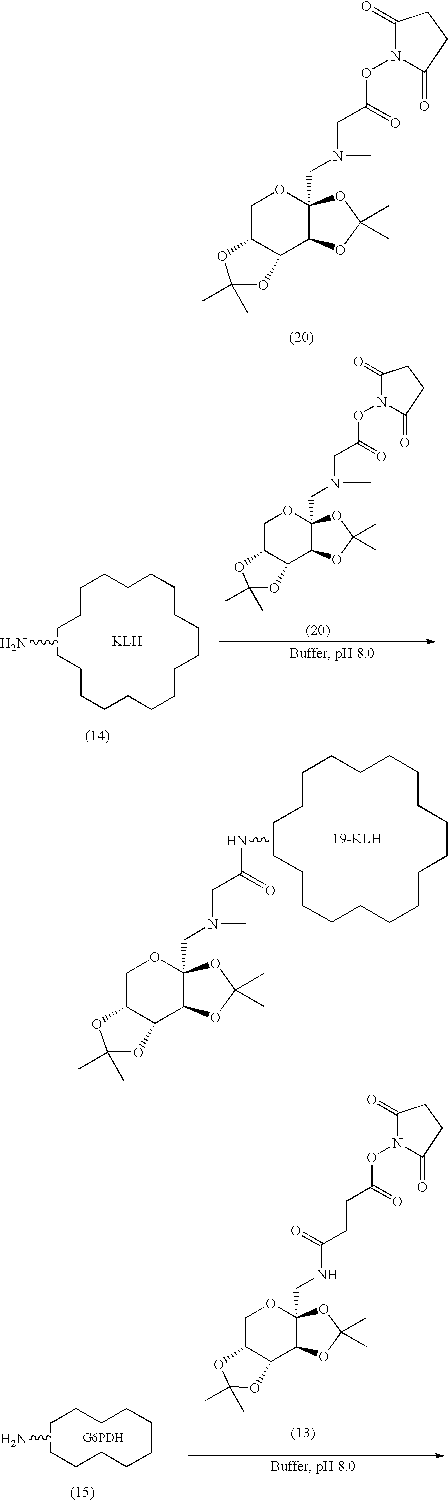

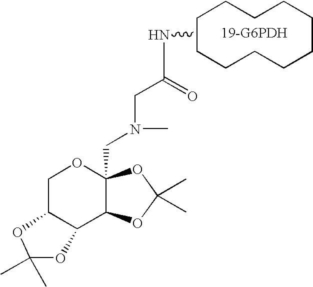

The diacetonefructose in dry pyridine was treated with methanesulfonyl chloride to produce diacetonefructose mesylate (17), which was treated with sarcosine benzyl ester to give compound (18). The compound (18) was hydrogenated with Palladium on 10% charcoal to give diacetonefructose derivative hapten (19), which was activated by EDC and NHS ester to give hapten (20). The activated hapten (20) was reacted with amines on KLH (14) and G6PDH (15) to give corresponding immunogen (19-KLH) and G6PDH conjugate (19-G6PDH) respectively.

The synthesis of hapten (22) and immunogen (22-KLH) is shown below:

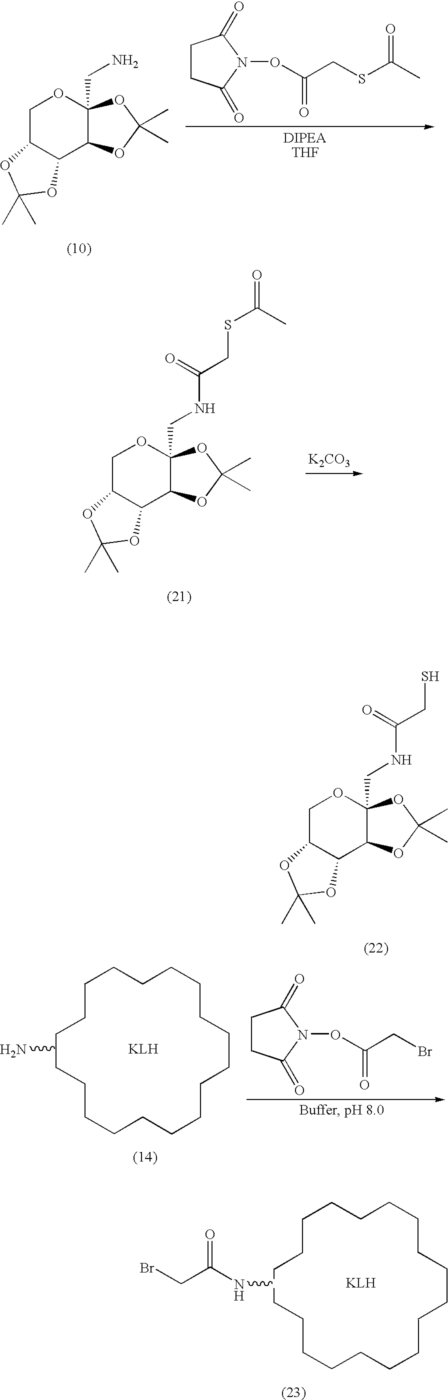

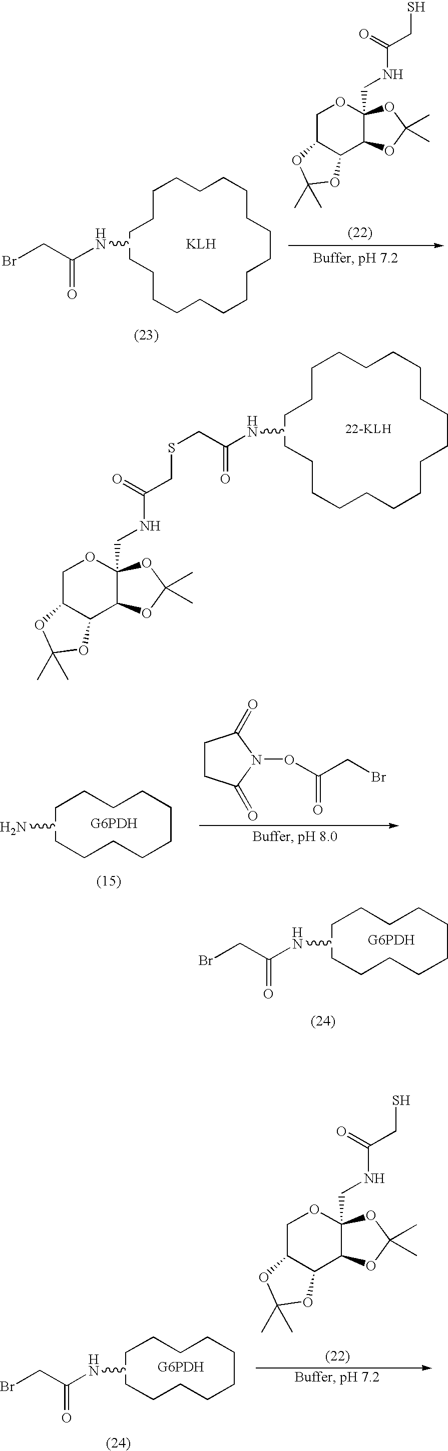

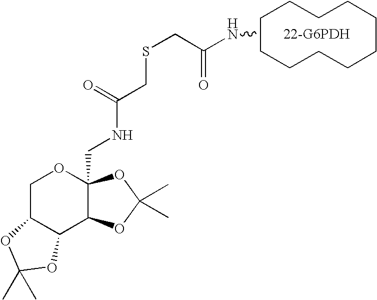

The thiol (—SH) chemistry was utilized to attach a diacetonefructose derivative to the protein. The amine-derivatized diacetonefructose (10) was treated with SATA (N-succinimidyl-S-acetylthioacetate) to produce hapten with protected sulfhydryl (21). The deprotection is performed by treating (21) with potassium carbonate to generate thioacetylated containing hapten (22). The keyhole limpet hemocyanin (KLH) is first treated with succinimidyl-bromo-acetate to introduce the bromo-acetamide group (23) for thiol modification, which is then reacted with thiol (22) in sodium phosphate buffer, pH=7.2 to give the desired immunogen (22-KLH). Similar procedure was used to prepare G6PDH conjugate (22-G6PDH).

The diacetinefructose derivative hapten (25) was designed for proteins containing cysteine groups such as mutant G6PDH. See, U.S. Pat. Nos. 6,455,288, 6,090,567, 6,033,890, which are incorporated by reference in their entireties. The synthesis of haptens (25) is shown below:

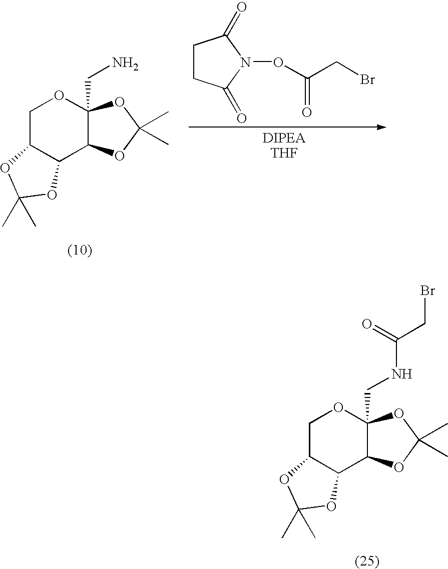

Acylation of diacetonefructose derivative (10) with bromoacetic N-hydroxyl succinimide under basic condition gave hapten (25). Hapten (25) was conjugated to a mutant enzyme (26) in phosphate buffer, pH 7.2 to give G6PDH conjugate (25-G6PDH).

The diacetonefructose derivative hapten (25) was conjugated to proteins where thiol groups were chemically introduced. The synthesis of diacetonefructose derivative immunogen (25-KLH) is shown in Example 12. Commercially available linker, N-Succinimidyl-S-acetylthioacetate was reacted with primary amine of KLH (14), which added protected sulfhydryls (27). Deprotection of protected sulfhydryls with hydroxyl amine produced the desired thiolated KLH (28). Conjugation of hapten (25) with thiolated KLH (28) resulted in immunogen (25-KLH).

All the prepared haptens have utility for KLH and G6PDH bio-conjugation. immunogens (10-KLH, 12-KLH, 12-L-KLH, 19-KLH, 22-KLH and 25-KLH) were used to raise antibodies. In an enzyme-based assay format, antibodies showed good reactivity with both diacetonefructosefructose and topiramate. The fact that all immunogens (10-KLH and 25-KLH) successfully raised antibodies and showed potential use in an enzyme-based topiramate immunoassay indicates that diacetonefructose derivatives facilitates well with the immunization process.

Exemplary embodiments are summarized herein below.

In an exemplary embodiment, the compound of the invention has the structure:

T-Y-Z¹

wherein T has the structure:

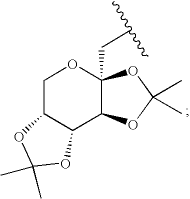

Y is a linker selected from a bond, R^{1a}, OR^{1a}, SR^{1a}, SOR^{1a}, SOOR^{1a}, SOONR^{1a}R^{1b} and NR^{1a}R^{1b} wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and Z¹ is a reactive functional group; with the proviso that Y-Z¹ does not comprise sulfamate.

In an exemplary embodiment according to the above paragraph, the reactive functional group is a member selected from an electrophilic group and a nucleophilic group.

In an exemplary embodiment according to any of the above paragraphs, the electrophilic group is a member selected from activated ester, acyl azide, acyl halide, acyl nitrile, aldehyde, alkyl halide, alkyl sulfonate, anhydrides, aryl halide, aziridine, boronate, caroxylic acid, carbodiimides, diazoalkane, epoxide, haloacetamide, halotriazine, imidoester, isocyanate, isothiocyanate, ketone, maleimide, phosphoramidite, silyl halide, sulfonate ester and sulfonyl halide.

In an exemplary embodiment according to any of the above paragraphs, the nucleophilic group is a member selected from alcohol, amine, aniline, carboxylic acid, glycol, hydrazine, hydroxylamine, phenol and thiol.

In an exemplary embodiment according to any of the above paragraphs, the reactive functional group is a member selected from amine, ester, thioester, thioether, halogen, isocyanate, isothiocyanate, thiol, imidoester, anhydride, maleimide, thiolacetone, diazonium groups, aldehyde, succinimide, hydroxysuccinimide, imidate, tosylate, triflate, mesylate and imidazole.

In an exemplary embodiment according to any of the above paragraphs, Y-Z¹ is NH₂.

In an exemplary embodiment according to any of the above paragraphs, Y-Z¹ is an activated hydroxyl group selected from tosylate, triflate, mesylate and —O—imidazole.

In an exemplary embodiment, the compound of the invention has the structure:

[T-Y]ᵣ—R²-Z²

wherein T has the structure:

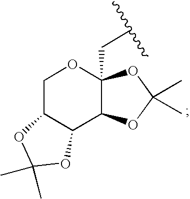

Y is a linker selected from a bond, R^{1a}, OR^{1a}, SR^{1a}, SOR^{1a}, SOOR^{1a}, SOONR^{1a}R^{1b}, NR^{1a}R^{1b}, wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; R² is a member selected from —NHCO—, —NHCONH—, —NHCSNH—, —NHOCO—, —S—, —NH(C═NH)—, —N═N— and —NH—; Z² is a member selected from an immunogenic carrier and a signal generating moiety; and r is an integer selected from 1 to the number of T-Y binding sites on Z²; with the proviso that Y-Z² does not comprise sulfamate.

In an exemplary embodiment according to any of the above paragraphs, the immunogenic carrier is a member selected from hemocyanin, globulin, albumin, and polysaccharide.

In an exemplary embodiment according to any of the above paragraphs, the albumin is a member selected from bovine serum albumin (BSA) and human serum albumin (HSA) and the hemocyanin is keyhole limpet hemocyanin (KLH).

In an exemplary embodiment according to any of the above paragraphs, the signal generating moiety is a member selected from a polypeptide, a polysaccharide, a synthetic polymer, an enzyme, a fluorogenic compound and a chemiluminescent compound.

In an exemplary embodiment according to any of the above paragraphs, the enzyme is a member selected from dehydrogenase, phosphatase, galactosidase and peroxidase.

In an exemplary embodiment according to any of the above paragraphs, the dehydrogenase is glucose-6-phosphate dehydrogenase (G6PDH), the phosphatase is alkaline phosphatase, the galactosidase is B-galactosidase and peroxidase is horseradish peroxidase.

In an exemplary embodiment according to any of the above paragraphs, Y comprises a backbone of 2-8 atoms that are members independently selected from C, O, S, N, P and halogen.

In an exemplary embodiment according to any of the above paragraphs, Y has the structure

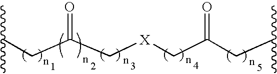

wherein each n₁, each n₃, each n₄ and each n₅ is independently selected from 0 to 10; n₂ is an integer selected from 0 and 1; and X is a member selected from S, O, NR³ and a bond wherein R³ is a member selected from H and substituted or unsubstituted alkyl.

In an exemplary embodiment according to any of the above paragraphs, Y is a member selected from —(CH₂)ₙC(O)—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, and —(CH₂)ₙNHC(O)—; and n is an integer selected from 0 to 10.

In an exemplary embodiment according to any of the above paragraphs, n is an integer selected from 1 and 2.

In an exemplary embodiment according to any of the above paragraphs, Y is —NR⁴R⁵— wherein R⁴ is selected from H, substituted or unsubstituted alkyl and substituted or unsubstituted heteroalkyl; and R⁵ is selected from substituted or unsubstituted alkyl and substituted or unsubstituted heteroalkyl.

In an exemplary embodiment according to any of the above paragraphs, Y is a member selected from —NHCOCH₂—, —NHCO(CH₂)₂—, —NHCO(CH₂)₂CONH(CH₂)₅—, —NHCOCH₂SCH₂— and —N(CH₃)CH₂—.

In an exemplary embodiment according to any of the above paragraphs, Z¹ is a member selected from —COOR⁴ and —SR⁵, wherein R⁴ and R⁵ are members each independently selected from H, halogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In an exemplary embodiment according to any of the above paragraphs, R⁴ is a member selected from H, Br, succinimidyl and phenylmethyl; and R⁵ is a member selected from H and —COCH₃.

In an exemplary embodiment according to any of the above paragraphs, R² is selected from —CONH— and —S—; and Z² is a member selected from KLH and G6PDH.

In an exemplary embodiment according to any of the above paragraphs, there is a proviso that Y-Z¹ or Y—R²-Z² does not comprise a member selected from sulfonamide, sulfonyl and sulfidyl.

In an exemplary embodiment according to any of the above paragraphs, the method of making an antibody comprises administering to a subject a compound according to any of the above paragraphs.

In an exemplary embodiment according to any of the above paragraphs, the antibody is generated by administering to a subject a compound according to any of the above paragraphs.

In an exemplary embodiment according to any of the above paragraphs, the antibody specifically binds to 2,3:4,5-bis-O-methylethylidene.

In an exemplary embodiment according to any of the above paragraphs, the antibody binds topiramate and has less than about 10% cross-reactivity with a member selected from a drug and a metabolite of topiramate wherein the drug is not topiramate.

In an exemplary embodiment according to any of the above paragraphs, the method of determining an amount of topiramate in a sample comprises (a) contacting the sample with an antibody raised against a compound according to any of the above paragraphs, thus yielding an antibody-topiramate complex; and (b) detecting the antibody-topiramate complex.

In an exemplary embodiment according to any of the above paragraphs, the method further comprises contacting the sample with a ligand competitively binding to the antibody.

In an exemplary embodiment according to any of the above paragraphs, the ligand is a compound according to any of the above paragraphs.

In an exemplary embodiment according to any of the above paragraphs, the antibody binds topiramate and has less than about 10% cross-reactivity with a member selected from a drug and a metabolite of topiramate, wherein the drug is not topiramate.

In an exemplary embodiment according to any of the above paragraphs, the drug is a member selected from an antiepileptic drug, an antiinflammatory drug, an anticonvulsive drug, and an antibacterial sulfonamide, wherein the drug is not topiramate.

In an exemplary embodiment according to any of the above paragraphs, the antiepileptic drug is phenytoin, the antiinflammatory drug is ibuprofen and the anticonvulsive drug is tiagabine.

In an exemplary embodiment according to any of the above paragraphs, the metabolite is a member selected from 9-hydroxytopiramate, 10-hydroxytopiramate, 2,3-diol-topiramate and 4,5-diol-topiramate.

In an exemplary embodiment, the kit for determining the amount of topiramate in a sample comprises an antibody raised against a compound according to any of the above paragraphs and ancillary reagents.

In an exemplary embodiment according to any of the above paragraphs, the kit further comprises a compound according to any of the above paragraphs.

In an exemplary embodiment according to any of the above paragraphs, the kit comprises instructions for using the kit.

### Example 1

### Preparation of Compound 10

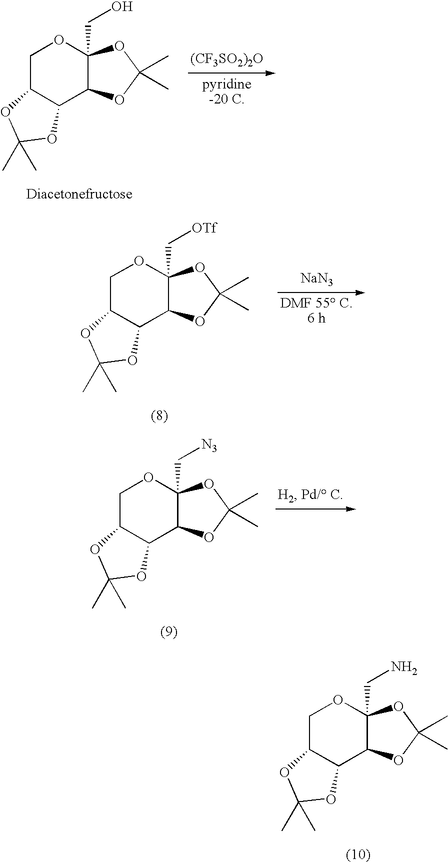

3 g of diacetonefructose (commercially available from sources such as Davos Chemical Corp., Upper Saddle River, N.J.; Tianyu Fine Chemical Co. Ltd., Shandong, China; and Carbopharm GmbH, Lannach, Austria) and pyridine 5.58 ml (6 equiv) in dichloromethane were treated with triflic anhydride 5.82 ml (3 equiv) at −20° C., and the mixture was stirred for 2 h. The crude triflate was purified by column chromatography to give 3.2 gm (71% yield) of oily product (8), which was dissolved in 10 ml of dimethylformamide and treated with sodium azide 0.58 g (1.1 equiv) at 55° C. for 6 h. Crude azide (9) in ethanol was hydrogenated in the presence of 0.48 g Pd/C, and acid/base workup gave 1.5 g of amine (10).

### Example 2

### Preparation of Compound 12

In a 100 mL round bottom flask, a solution of 1 g (3.86 mmol) of (10) in 10 mL tetrahydrofuran (anhydrous) is combined with 1 mL (5.75 mmol) N,N-diisopropylethylamine (DIPEA), and stirred under argon. 0.62 g 9 (6.2 mmol) of succinic anhydride and 25 mg (0.20 mmol) of 4-dimethylaminopyridine (DMAP) are added to the above solution to form a reaction mixture. The reaction mixture is stirred under argon for 12 hours, and the solvent is evaporated under reduced pressure to form a residue. The residue is purified by flash column chromatography with ethyl acetate as the eluent. The fractions containing the succinyl derivative (12) are combined and concentrated to yield about 0.98 g (70% yield) of the final product.

### Example 3

### Preparation of Compound 19

To a stirred solution of diacetonefructosefructose (2.60 g, 10 mmol) in dry pyridine (50 ml) at ice bath temperature and an argon atmosphere was added dropwise a solution of methanesulfonyl chloride (1.26 g, 11 mmol) in such a rate that temperature did not exceed 10° C. After the addition was completed, the reaction was stirred for one hour and then the ice bath was removed. The reaction was allowed to stir at RT for 5 hours or until no starting material was detected (TLC, silica gel, CH₂Cl₂: MeOH, 95:5). The solvent was then removed at RT under high vacuum to a yellow oil. The oil was then purified on a silica gel column (CH₂Cl₂: MeOH 95:5) to give the 2.88 g (85% yield) of pure diacetonefructose mesylate (17) as a white solid.

To a stirred solution of (17) (1.77 g, 5.2 mmol) in anhydrous THF (20 ml) and under an atmosphere of argon was added a solution of sarcosine benzyl ester (1.86 g, 10.4 mmol) and imidazole (0.71 g, 10.4 mmol) in dichloromethane (10 ml) over a period of 1 hour. After the addition was complete, the mixture was allowed to stir overnight. TLC (silica gel, ethyl acetate) detected no starting material. To the reaction was added dichloromethane (30 ml) and the reaction was washed with brine (50 ml, 2×) and water (50ml, 2×). The organic phase was then separated and dried (MgSO₄) and filtered. The filtrate was then evaporated to dryness to give crude (18) as thick oil. The oil was then purified on a column (silica gel, ethyl acetate) to give compound (18) as a white solid (1.48 g, 3.5 mmol) in 67% yield.

To a stirred solution of (18) (1.09 g, 2.6 mmol) in anhydrous THF (20 ml) was added Palladium on % 10 charcoal (40 mg). The reaction mixture was then purged with argon and was hydrogenated at atmospheric pressure over night. The mixture was carefully filtered through Celite. The filtrate was then evaporated to dryness under vacuum to give 649 mg (1.96 mmol) of the desired compound (19) as a white solid in 75% yield.

### Example 4

### Preparation of Compound 22

To a stirred solution of (10) 900 mg, 3.47 mmol) in THF (10 ml) was added diisopropylethylamine (2.42 ml, 13.88 mmol) N-Succinimidyl-S-acetylthioacetate (1.20 g, 5.2 mmol). The reaction mixture was stirred at room temperature for 3 hours. TLC analysis of the mixture showed that a new spot as a product in comparison with (10). The organic solvent was removed to dryness by rotary evaporation under reduced pressure. The residue was purified by flash column chromatography (silica gel) using ethyl acetate/hexane (7/3) as the eluent to give the desired product (21) (912 mg, 70% yield).

To a solution of (21) (118 mg, 0.315 mmol) in degassed (N₂) MeOH (5 mL) and H₂O (0.2 ml) was added K₂CO₃ (87 mg, 0.630 mmol) under nitrogen. The reaction mixture was stirred at room temperature under nitrogen for 1 hour. TLC analysis of the mixture showed that starting material (21) had disappeared and a new spot was formed as a product (silica gel, MeOH/CH₂Cl₂=1/9, I₂, Ellman's reagent). MeOH was filtered to remove excess K₂CO₃ and the filtrate was concentrated by rotary evaporation in room temperature. The residue was dried under high vacuum for 0.5 hour at room temperature to give the desired hapten (22). The activated hapten (22) was dissolved in DMF (0.8 ml) for the next reaction.

### Example 5

### Preparation of Diacetonefructose Derivative (25)

To a solution of compound (10) (40 mg, 0.154 mmol) in tetrahydrofuran (anhydrous) (10 mL) were added N,N-diisopropylethylamine (53 μL, 39 mg, 0.302 mmol) and a solution of bromoacetic N-hydroxyl succinimide (40.5 mg, 0.174 mmol) in tetrahydrofuran (5 mL) at 0° C. under argon. The reaction was stirred at room temperature for 2 hours. Water (10 mL) was added and most of tetrahydrofuran was removed by rotary evaporation. The aqueous phase was extracted with CH₂Cl₂ (3×30 mL). The combined organic phase was washed with water (15 mL) and dried over MgSO4. The organic phase was filtered and concentrated to dryness. The residue was purified by flash column chromatography (silica gel) using ethyl acetate/hexane (⅔) as an eluent to give compound (25) (20 mg, 34%).

### Example 6

### Preparation Immunogen 10-KLH

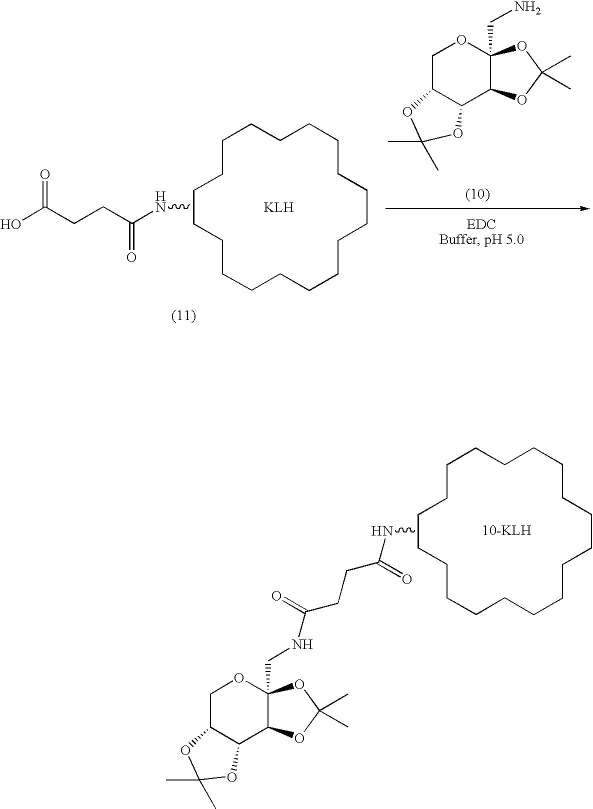

Lyophilized succinylated KLH (11) (Sigma, 11 mg) is reconstituted with 2 mL deionized water. The KLH solution is dialyzed overnight two changes (2.0 L each) MES buffer (0.1 M MES, 0.9 M NaCl, 0.02% NaN₃, pH 4.7). After dialysis 6 mg of succinylated KLH (11) is transferred to a reaction vial. Compound (10) (3.7 mg, 11.1 μM) is dissolved in dry DMF and added to the reaction vial slowly. EDC (Pierce, 10 mg) is dissolved in 1 mL deionized water and immediately add 50 μL of this KLH solution. Additional EDC aliquots (10 μL per addition) are added until slight precipitation occurred during the conjugation reaction. The reaction is allowed to proceed for approximately 2 h under constant mixing at room temperature to give immunogen (10-KLH). The reaction mixture is then dialyzed against three changes (2.0 L each) of HEPES buffer (0.05 M, pH 7.2, 1 mM EDTA).

### Example 7

### Preparation Immunogen 12-KLH and Immunogen 19-KLH

This conjugation technique is generally applicable to all derivatives which are conjugated through a carboxylic acid moiety. The hapten is activated upon conversion of the carboxylic acid moiety to N-hydroxysuccinimide (NHS) ester.

### Activation of Diacetonefructose Derivative (12)

To a stirred solution of (12) (10.7 mg, 0.03 mmol) in dried DMF (0.5 mL) is added 1-ethyl-3-(3-dimethylamino propyl)carbodiimide (EDAC) (18 mg, 0.094 mmol) and N-hydroxysuccinimide (NHS) (11.68 mg, 0.102 mmol) at ice bath temperatures. The mixture is stirred overnight to form compound (13). Ester formation is monitored by TLC analysis. Similar procedure was used to activate diacetonefructose derivative (19).

### Preparation Immunogen 12-KLH and Immunogen 19-KLH

Two vials of lyophilized KLH (Pierce, 27 mg per vial) are reconstituted with 2 mL of deionized water each and pooled. The mixture is allowed to stand overnight at 4° C. A buffer exchange is done by dialyzing overnight the KLH solution against 2 L of sodium bicarbonate buffer (0.1 M, pH 8.9). The final volume of the KLH preparation is 3.75 mL at a concentration of 14.4 mg/mL. A 1.2 mL aliquot of the KLH preparation (17.28 mg) is transferred into a reaction vial. The solution of (13) (320 μL) is then added slowly (10-20 μL per addition) to the solution of KLH (14) over a period of 2 h at ice bath temperatures. After the addition is completed, the mixture is stirred in a 4° C. cold room overnight. This solution is then dialyzed against three changes (2.0 L each) of HEPES buffer (10 mM, pH 7.0, 1 mM). The final concentration of the immunogen (12-KLH) preparation is 4.5 mg/mL. Similar procedure was used to prepare immunogen (19-KLH). The Immunogen (12-KLH) was used for the immunizations.

### Example 8

### Preparation Immunogen 12-L-KLH

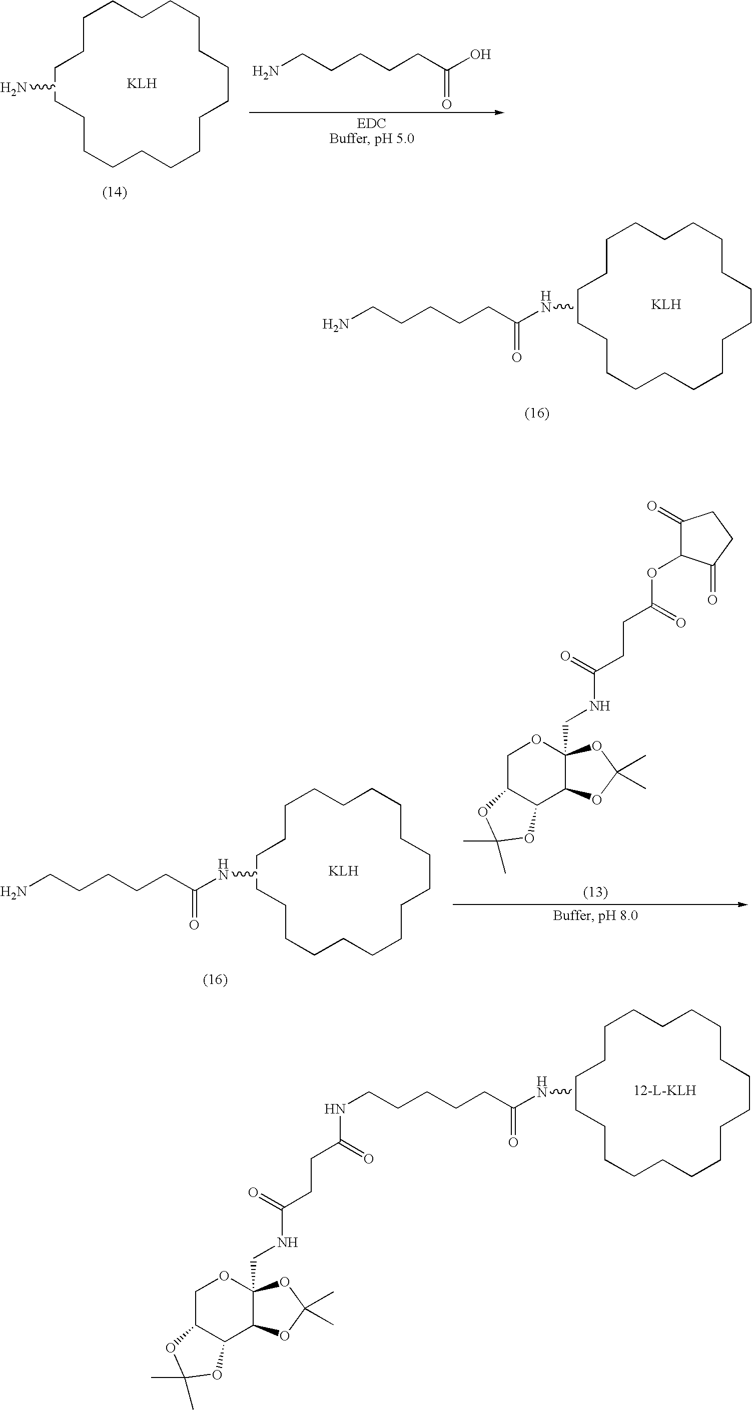

A solution of 6-aminocaproic acid (0.30 g), KLH (50 mg) and dimethylamino-propyl-3-ethylcarboiimide hydrochloride (1.0 g) in water (50 mL), adjusted to pH=5 by 0.1 M MES buffer, was stirred for 48 hours to give KLH (16). Conjugation of diacetonefructose derivatives (12) was performed as described in Example 7. The Immunogen (12-L-KLH) was used for the immunizations.

### Example 9

### Preparation of Bromoacetyl KLH (23)

To a solution of KLH (20 mg) in NaH₂PO₄—Na₂HPO₄ buffer (pH=8.0, 0.1M, 2.0 mL) at 4° C. (ice-bath) was added a solution of bromoacetic acid NHS ester (5.8 mg, 0.024 mmol) in DMF (0.2 mL). The pH value was maintained at 8.0. The reaction mixture was stirred in the cold-room (4° C.) for 16 hours. The mixture was purified by a Sephadex G-50 column, eluting with NaH₂PO₄—Na₂HPO₄ buffer (pH=7.00, 0.025 M). The eluted fractions from the column were monitored by UV at 280 nm. A clean separation between bromoacetyl-KLH and the hapten was obtained. Fractions containing the product were pooled together (8.0 mL) and concentrated to 3.0 mL of bromoacetyl-KLH (23) by an Amicon concentrator for the next reaction.

### Example 10

### Preparation Immunogen 22-KLH

To a solution of bromoacetyl-KLH (23) (50 ml, pH=8.00) was added the diaceteonefructose derivative (22) solution slowly at 4° C. under nitrogen. The pH value was maintained at 8.0. The reaction was stirred at 4° C. (cold room) for 16 hours. The reaction mixture was separated using a Sephadex G-25 column equilibrated with NaH₂PO₄—Na₂HPO₄ buffer (pH=7.0, 0.1 M). The UV detector at 280 nm monitored the eluted fractions from the column. A clean separation between KLH immunogen and the hapten was obtained. Fractions containing protein were pooled to a total volume of 180 ml and concentrated to 105 ml. The concentration of immunogen (22-KLH) was measured by using BCA Protein Concentration Assay.

### Example 11

### Preparation of Thiolated KLH (28)

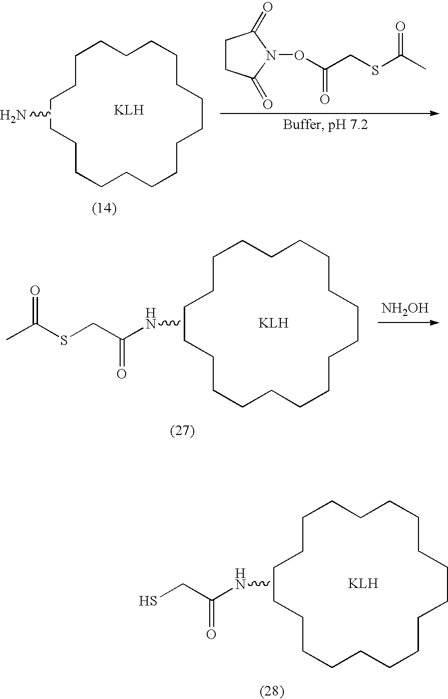

One vial of lyophilized KLH (Pierce, 21 mg) was reconstituted with 3 mL of phosphate buffer (0.1 M, 0.15 M NaCl, 1 mM EDTA, pH 8.0). The KLH (14) solution was transferred to a reaction vial. Immediately before reaction, 6-8 mg of SATA (N-Succinimidyl-S-acetylthioacetate) was dissolved in 0.5 mL of DMSO (results in ˜55 mM solution). Thirty μl of the SATA solution was combined with 3.0 mL of protein solution (7 mg/mL). The contents were mixed and reaction incubated at room temperature for at least 30 minutes. A Sephadex G-50 column was equilibrated with two column volumes of buffer (0.1 M phosphate, 0.15 M NaCl, pH 7.2-7.5). The reaction mixture was appllied to column. Fraction (500 μL) were collected immediately. The fractions that contain protein were identified by measuring absorbance at 280 nm. Protein fractions were pooled to give 12 mL. Deacylation to generate a sulfhydryl for use in cross-linking was accomplished adding 1.2 mL deacetylation solution (0.5 M Hydroxylamine, 25 mM EDTA in PBS, pH 7.2-7.5). Contents were mixed and reaction incubated for 2 hours at room temperature. Sephadex G-50 desalting column was used to purify the sulfhydryl-modified protein from the hydroxylamine in the deacetylation solution. The pooled fraction were concentrated to 2.6 mL (8 mg/mL) using Amicon concentrator.

### Example 12

### Preparation Immunogen 25-KLH

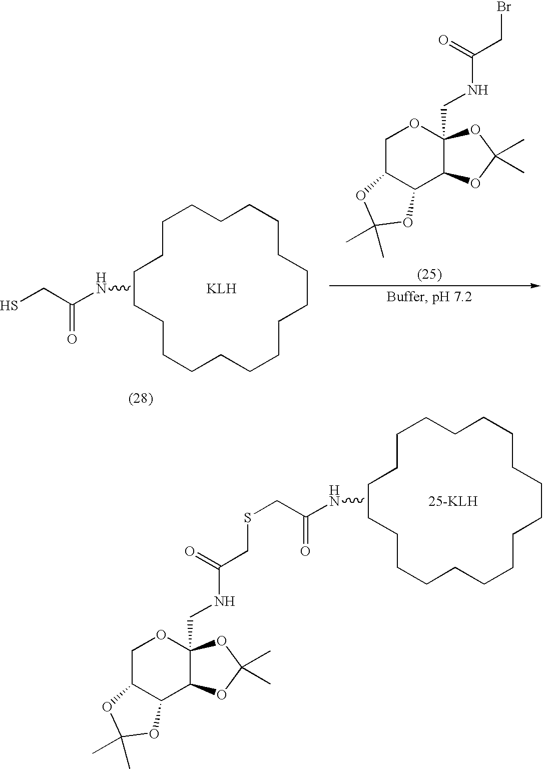

Dithiothreitol (DTT, 1 mM) was added to thiolated KLH (28) to ensure reduction of disulfide bonds. The solution was allowed to mix overnight at 4° C. 10.2 mg bromoacetamido diacetonefructose derivative hapten (25) was dissolved in 0.2 mL DMF. Diacetonefructose derivative hapten (25) DMF solution was added in 5 to 10 μL quantities to a solution of thiolated KLH (28). The reaction was continued overnight at 4° C. This solution was dialyzed against three changes (2.0 liter each) of HEPES buffer (10 mM, pH 7.0, 1 mM EDTA). This procedure yielded immunogen (25-KLH).

### Example 13

### Preparation Glucose-6-Phosphate Dehydrogenase Conjugate 12-G6PDH

Lyophilized G6PDH (Worthington Biochem. Corp., 42.2 mg) is reconstituted with 3.5 mL deionized water to give a solution of 12.1 mg/mL. The mixture is allowed to stand overnight at 4° C. The mixture is then dialyzed overnight at 4° C. against 2 L of sodium bicarbonate buffer (0.1 M, pH 8.9). After dialysis, 0.6 mL (7.2 mg) of enzyme solution is transferred to a reaction vial.

Activated product compound (13) from Example 7 was added in 5 to 10 μL quantities to a solution of glucose-6-phosphate dehydrogenase (G6PDH, 0.1 M in sodium carbonate buffer) glucose-6-phosphate (G6P, 4.5 mg/mg G6PDH), and NADH (9 mg/mg G6PDH) in a pH 8.9 sodium carbonate buffer at ice bath temperature. After the addition of each portion of solution of compound (13) a 2 μL aliquot is taken and diluted 1:500 with enzyme buffer. A 3 μL aliquot of this diluted conjugation mixture can be assayed for enzymatic activity similar to that described in Example 17 below. The reaction is monitored and stopped at approximately 65% deactivation of enzyme activity. The mixture is desalted with a PD-10 pre-packed Sephadex G-25 (Pharmacia, Inc.) and pre-equilibrated with HEPES buffer (10 mM, pH 7.0, 1 mM EDTA). The reaction mixture is applied to the column and the protein fractions pooled. The pooled fractions are dialyzed against three (1.0 L each) changes of HEPES (10 mM, pH 7.0, 1 mM EDTA) to yield a solution of conjugate (12-G6PDH). Similar procedure was used for conjugation of diacetonefructose (19) glucose-6-phosphate dehydrogenase to form (19-G6PDH).

### Example 14

### Preparation of Bromoacetyl Glucose-6-Phosphate Dehydrogenase (24)

100 μL DMF is added to bromoacetic acid NHS (Sigma 3.06 mg, 12.97 μM) and stirred. A 2.0 mL (10 mg/mL) G6PDH solution is prepared in 0.025 M phosphate carbonate buffer, pH 7.2 and adjusted to pH 8.5 with 0.4 M carbonbate buffer. 45 mg disodium G6P and 90 mg NADH, is dissolved in the G6PDH solution. Bromoacetic acid NHS is added to G6PDH solution at 5 μL increments. Enzyme activity is measured on the HITACHI 917 analyzer after each addition. Bromoacetic acid NHS is added until approximately 63.0% enzyme deactivation was obtained. G6PDH conjugation solution is dialyzed with 3×4 liter portions of 0.01 M phosphate, pH 7.2.

### Example 15

### Preparation Glucose-6-Phosphate Dehydrogenase Conjugate 22-G6PDH

Bromoacetyl Glucose-6-Phosphate Dehydrogenase (24) was buffer exchanged with 50 mM phosphate-1.0 mM EDTA, pH 7.25. A solution of the protein (2 mL at 5 mg/mL) was then mixed with a dithioerythreitol (25 mM final concentration in the phosphate-EDTA buffer) and mixture incubated at 4° C. for 16 hours. The protein solution was then buffer exchanged with 50 mM phosphate, 1.0 mM EDTA, 5 mM DTT, pH 7.25. The protein solution (2 mL at 5 mg/mL) was mixed with 40 fold molar excess of a DMF solution (0.05 mL) of diacetonefructose derivative (22) and reaction mixture stirred gently at 4° C. for 16 to 24 hours. Excess (22) was separated from the enzyme-hapten conjugate by passing the reaction mixture over a column of Sephadex G 50 in 50 mM phosphate, pH 7.0. The column fractions containing the enzyme-hapten conjugate were pooled by measuring absorption at 280 nm which gave conjugate (22-G6PDH).

### Example 16

### Preparation Glucose-6-Phosphate Dehydrogenase Conjugate 25-G6PDH

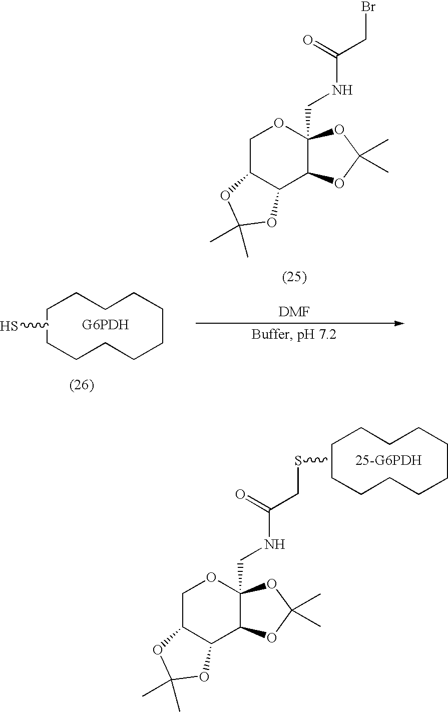

SH-G6PDH was buffer exchanged with 50 mM phosphate-1.0 mM EDTA, pH 7.25. A solution of the enzyme (2 mL at 5 mg/mL) was mixed with a solution of dithioerythreitol (20 μL of a 0.5 M solution in the phosphate-EDTA buffer) and mixture incubated at 4° C. for 16 hours. The protein solution was then buffer exchanged with 50 mM phosphate-1.0 mM EDTA-0.025 mM DTT, pH 7.25. Thiol content of the protein were determined by titration with a solution of dithiodipyridine, and reported as thiols per mole of the protein. The protein solution (2 mL at 5 mg/mL) was mixed with 40 fold molar excess of a DMF solution (0.05 mL) of hapten (25) and reaction mixture stirred gently at 4° C. for 16-24 hours. Excess hapten (25) was separated from the enzyme-hapten conjugate by passing the reaction mixture over a column of Sephadex G 50 in 50 mM phosphate, pH 7.0. The column fractions containing the enzyme-hapten conjugate were pooled by measuring absorption at 280 nm to give conjugate (25-G6PDH).

### Example 17

### Preparation of Diacetonefructose Derivative Polyclonal Antibodies Reactive to Topiramate

The topiramate antibodies and enzyme conjugates (12-G6PDH), (19-G6PDH), (22-G6PDH) or (25-G6PDH) may be employed in assays for the detection of topiramate. Either of the immunogens (10-KLH), (12-KLH), (12-L-KLH), (19-KLH), (22-KLH), or (25-KLH) can be injected into a mouse, sheep or rabbit to raise antibody.

Polyclonal sera from 12 live rabbit were prepared by injecting six animals with immunogen (12-KLH) and another six animals with immunogen (12-L-KLH). This immunogenic formulation comprises 200 μg of the immunogen for the first immunization and 100 μg for all subsequent immunizations. Regardless of immunogen amount, the formulation was then diluted to 1 mL with sterile saline solution. This solution was then mixed thoroughly with 1 mL of the appropriate adjuvant: Freund's Complete Adjuvant for first immunization or Freund's Incomplete Adjuvant for subsequent immunizations. The stable emulsion was subsequently injected subcutaneously with a 19×1 ½ needle into New Zealand white rabbits. Injections was made at 3-4 week intervals. Bleeds of the immunized rabbits were taken from the central ear artery using a 19×1 needle. Blood was left to clot at 37° C. overnight, at which point the serum was poured off and centrifuged. Finally, preservatives were added in order to form the polyclonal antibody material. Rabbit polyclonal antibodies to topiramate produced by the above procedure immunized with immunogen (12-KLH) are designated as #11053, #11054, #11055, #11056, #11057, and #11058, and those immunized with immunogen (12-L-KLH) are designated as #11338, #11339, #11340, #11341, and #11342. Rabbit polyclonal antibody #11341 is used in examples below.

Rabbit polyclonal antibody #1134 was used to generate a calibration curve, and evaluate assay precision, accuracy and specificity. The antibody was added into the antibody diluent to prepare the antibody reagent. The antibody reagent consists of antibody as prepared above, buffer, stabilizers, preservatives, and the substrates for the enzyme conjugate NAD and glucose 6 phosphate. Enzyme conjugate comprising compound (12-G6PDH) G6PDH was added into the conjugate reagent to prepare the enzyme conjugate reagent. The enzyme conjugate reagent consists of the conjugate, buffer, stabilizers and preservatives. Enzyme conjugate (12-G6PDH) was used with rabbit polyclonal antibody #11341 in examples below. This technique is generally applicable to produce polyclonal antibodies to diacetonefructose derivatives and assess their utility.

### Example 18

### HITACHI 917 Clinical Chemistry Analyzer

The diacetonefructose derivative antibodies and enzyme conjugates may be advantageously used in a homogeneous assay format to detect topiramate in samples. An enzyme immunoassay or ARK Assay, which is a homogeneous enzyme immunoassay experiment, is performed to test the polyclonal antibodies prepared as in Example 17. The ARK Assay for topiramate is conducted using a liquid, ready-to-use, two-reagent kit. A clinical chemistry analyzer useful to set up the assay is HITACHI 917. The HITACHI 917 is an automated biochemistry analyser used by medical laboratories to process biological fluid specimens, such as urine, cerebrospinal fluid, and most commonly, blood. Manufactured by Boehringer Mannheim, the HITACHI 917 is a commonly used routine chemical bichromatic analyzer. Topiramate containing sample is incubated with antibody reagent followed by the addition of the enzyme conjugate reagent. The enzyme conjugate activity decreases upon binding to the antibody. The enzyme conjugate, which is not bound to the antibody, catalyzes the oxidation of glucose 6-phosphate (G6P). The oxidation of G6P is coupled with the reduction of NAD⁺ to NADH, which can be measured at 340 nm. The change in the absorbance at 340 nm can be measured spectrophotometrically. The topiramate concentration in a specimen can be measured in terms of G6PDH activity. The increase in the rate at 340 nm is due to the formation of NADH and is proportional to the enzyme conjugate activity. An assay calibration curve is generated using topiramate spiked into negative calibrator matrix (See Example 19 below). The assay rate increases with increasing the concentration of drug in the sample.

### Example 19

### Calibration Curve

Topiramate was dissolved in methanol to give a stock solution of 1000 μg/mL. Pooled human serum was aliquoted in 10 mL portions. Topiramate stock solution was added to the aliquots of human serum in preparing a series of known concentrations of topiramate calibrators ranging from 0 to 60 μg/mL. Similarly, Quality Control samples were prepared (2.0, 10.0 and 40.0 μg/mL). Antibody Reagent was prepared by adding antibody #11431 to antibody/substrate diluent. The antibody/substrate reagent was assayed with Enzyme Conjugate Reagent (12-G6PDH). The antibody/substrate reagent was assayed with Enzyme Conjugate Reagent (12-G6PDH). Calibration curves were generated on the HITACHI 917 automated clinical chemistry analyzer, as described in Example 18 by assaying each level in duplicate. An example of these calibrator rates is shown in Table 2.

TABLE 2
Calibrator Reaction Rate

| Topiramate Conc. | Reaction Rate (mA/min) |
|---|---|
| (μg/mL) | Average of Duplicates |
| 0.0 | 328.3 |
| 3.5 | 406.0 |
| 7.5 | 442.4 |
| 15.0 | 478.3 |
| 30.0 | 511.6 |
| 60.0 | 542.4 |

### Example 20

### Precision and Accuracy of the Measurement

Three topiramate Quality Control samples were prepared as described in Example 19 to give concentrations of topiramate of 2.0, 10.0 and 40.0 μg/mL. Enzyme Conjugate Reagent #110607-D63-E and Antibody Reagent #11341P4-6-Ab was used to generate precision data shown in Table 3. The precision data were derived from 2 runs on the same day. Each run consisted for generating a calibration curve and 10 replicates of each QC level per run with a total of 20 replicates from 2 runs. Quantification was performed on the HITACHI 917 analyzer as described in Example 18. The precision coefficient of variation (CV %) was calculated for each Quality Control data set (Table 3). Also, the accuracy of the measurement was calculated as a percentage of the nominal value of the QC samples (Table 3).

TABLE 3
Precision

| | QC Conc. | Mean | Precision | Accuracy |
|---|---|---|---|---|
| N | (μg/mL) | (μg/mL ± SD) | (CV %) | (%) |
| 20 | 2.0 | 1.97 ± 0.05 | 4.59 | 98.50 |
| 20 | 10.0 | 10.24 ± 0.35 | 3.44 | 102.40 |
| 20 | 40.0 | 40.09 ± 1.95 | 4.86 | 100.23 |

### Example 21

Interference of the immunoassay in the presence of potentially coadministered drugs was performed on the HITACHI 917. Interference of the immunoassay was evaluated by adding potentially coadministered drugs to human serum containing topiramate of 20.0 μg/mL and determining the increase in the apparent concentration as a result of the presence of coadministered drug. Separate stock solutions of topiramate were prepared by dissolving the drug in methanol to give a stock solution of 1000 μg/mL. A high concentration of each coadministered drug was spiked into normal human serum with containing topiramate 20 μg/mL and assayed. Each sample was assayed in duplicate. Testing was performed on the HITACHI 917 analyzer. The percentage concentration above 20.0 μg/mL of topiramate was calculated for each coadministered drug tested. No interference greater than 1% was observed with the coadministered drugs tested (Table 4).

TABLE 4
Summary of Coadministered Drug Interference Study

| | Amount of Drug | | |
|---|---|---|---|
| | Added to a Sample | | % Interference |
| | Containing | | [(20.0 μg/mL |
| | 20.0 μg/mL | | Topiramate) − |
| | Topiramate | Result | (Result)] ÷ |
| Cross Reactant | (μg/mL) | (μg/mL) | (drug conc.) × 100 |
| Acetominophen | 50.0 | 19.81 | −0.38 |
| Acetazolamide | 50.0 | 20.28 | 0.56 |
| Acetylsalicyclic | 100.0 | 20.25 | 0.25 |
| Acid | | | |
| Atenolol | 50.0 | 19.80 | −0.40 |
| Caffeine | 100.0 | 20.35 | 0.35 |
| Carbamazepine | 100.0 | 19.57 | −0.43 |
| Chlorthalidone | 100.0 | 20.16 | 0.16 |
| Clonazepam | 50.0 | 20.44 | 0.70 |
| Diazepam | 50.0 | 20.41 | 0.64 |
| Ethosuxamide | 500.0 | 19.57 | −0.09 |
| Famotidine | 5.0 | 19.99 | −0.2 |
| Felbamate | 500.0 | 21.09 | 0.20 |
| Furosemide | 10.0 | 20.01 | 0.10 |
| Gabapentin | 100.0 | 19.51 | −0.49 |
| Ibuprofen | 50.0 | 21.24 | 2.30 |
| Lamotrogine | 100.0 | 18.98 | −1.02 |
| Levetiracetam | 200.0 | 19.10 | −0.45 |
| Metoprolol | 100.0 | 20.44 | 0.35 |
| Phenytoin | 50.0 | 20.16 | 0.32 |
| Phenobarbitol | 100.0 | 21.08 | 1.08 |
| Primidone | 100.0 | 18.17 | −1.83 |
| Salicylic Acid | 750.0 | 19.57 | −5.73 |
| Sulfanilamide | 2000.0 | 18.77 | −0.16 |
| Tolbutamide | 750.0 | 19.39 | −0.08 |
| Valproic Acid | 200.0 | 20.99 | 0.50 |
| Verapamil | 100.0 | 21.16 | 1.07 |
| Zonisamide | 200.0 | 20.16 | 0.08 |

### Example 22

### Specificity of the Immunoassay in the Presence of Other 9-hydroxytopiramate, a Topiramate Metabolite.

The topiramate metabolites, 9-OH-topiramate, 10-OH-topiramate and were tested in the immunoassay for potential crossreactivity with antibody from rabbit #11341 which was immunized with immunogen (10-KLH). Each metabolite sample was assayed in duplicate on the HITACHI 917 in the presence of 20 μg/mL topiramate. The apparent topiramate concentration for the metabolite is the difference in concentration with metabolite minus the concentration without metabolite. As shown in Table 5, immunoassay using antibody from rabbit #11341 does not significantly crossreact with topiramate metabolites indicating a highly specific antibody has been produced.

TABLE 5
Crossreactivity to Topiramate Metabolites
Extremely high levels of topiramate metabolites were tested.

| | Metabolite | Apparent | |
|---|---|---|---|
| | Concentration | Topiramate | % Cross |
| Metabolite | (μg/mL) | (μg/mL) | Reactivity |
| 9-OH-Topiramate | 50.0 | 1.59 | 3.18 |
| 10-OH-Topiramate | 50.0 | 1.60 | 3.20 |
| 4,5-diol-Topiramate | 50.0 | 0.38 | 0.76 |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

All publications, patent applications and issued patents cited herein are incorporated by reference in their entirety for all purposes. The singular articles “a” and “an” mean “at least one”.

We claim:

1. A compound having the structure:
T-Y-Z¹
wherein T has the structure: 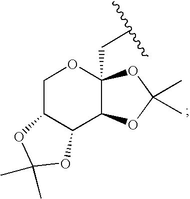
Y is a linker selected from a bond, R^{1a}, OR^{1a}, SR^{1a}, SOR^{1a}, SOOR^{1a}, SOONR^{1a}R^{1b} and NR^{1a}R^{1b}
wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and
R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and
Z¹ is a reactive functional group;
with the proviso that Y-Z¹ does not comprise sulfamate.

2. A compound having the structure:
[T-Y]ᵣ—R²-Z²
wherein T has the structure: 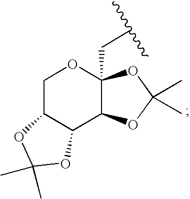
Y is a linker selected from a bond, R^{1a}, OR^{1a}, SR^{1a}, SOR^{1a}, SOOR^{1a}, SOONR^{1a}R^{1b}, NR^{1a}R^{1b},
wherein R^{1a} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and
R^{1b} is a member selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl;
R² is a member selected from —NHCO—, —NHCONH—, —NHCSNH—, —NHOCO—, —S—, —NH(C═NH)—, —N═N—and —NH—;
Z² is a member selected from an immunogenic carrier and a signal generating moiety; and
r is an integer selected from 1 to the number of T-Y binding sites on Z²;
with the proviso that Y-Z² does not comprise sulfamate.

3. The compound of claim 1 or 2 with the proviso that Y-Z¹ or Y—R²-Z² does not comprise a member selected from sulfonamide, sulfonyl and sulfidyl.

4. The compound of claim 1 or 2 wherein Y has the structure 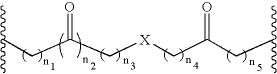
wherein
each n₁, each n₃, each n₄ and each n₅ is independently selected from 0 to 10;
n₂ is an integer selected from 0 and 1; and
X is a member selected from S, O, NR³ and a bond wherein R³is a member selected from H and substituted or unsubstituted alkyl.

5. The compound of claim 1 or 2 wherein Y is —NR⁴R⁵— wherein R⁴ is selected from H, substituted or unsubstituted alkyl and substituted or unsubstituted heteroalkyl; and R⁵ is selected from substituted or unsubstituted alkyl and substituted or unsubstituted heteroalkyl.

6. The compound of claim 5 wherein Y is a member selected from —NHCOCH₂—, —NHCO(CH₂)₂—, —NHCO(CH₂)₂CONH(CH₂)₅—, —NHCOCH₂SCH₂— and —N(CH₃)CH₂—.

7. The compound of claim 1 wherein the reactive functional group is a member selected from amine, ester, thioester, thioether, halogen, isocyanate, isothiocyanate, thiol, imidoester, anhydride, maleimide, thiolacetone, diazonium groups, aldehyde, succinimide, hydroxysuccinimide, imidate, tosylate, triflate, mesylate and imidazole

8. The compound of claim 6 wherein Z¹ is a member selected from —COOR⁴ and —SR⁵, wherein R⁴ and R⁵ are members each independently selected from H, halogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

9. The compound of claim 8 wherein R⁴is a member selected from H, Br, succinimidyl and phenylmethyl; and R⁵ is a member selected from H and —COCH₃.

10. The compound of claim 6 wherein R2 is a member selected from —CONH— and —S—; and Z² is a member selected from KLH and G6PDH.

11. The compound of claim 2 wherein the immunogenic carrier is a member selected from hemocyanin, globulin, albumin, and polysaccharide.

12. The compound of claim 2 wherein the signal generating moiety is a member selected from a polypeptide, a polysaccharide, a synthetic polymer, an enzyme, a fluorogenic compound and a chemiluminescent compound.

13. The compound of claim 12 wherein the enzyme is a member selected from dehydrogenase, phosphatase, galactosidase and peroxidase.

14. A method of making an antibody comprising administering to a subject the compound of 2.

15. An antibody generated by administering to a subject the compound of claim 2.

16. The antibody of claim 15 wherein the antibody specifically binds to 2,3:4,5-bis-O-methylethylidene.

17. An antibody that binds topiramate and has less than about 10% cross-reactivity with a member selected from a drug and a metabolite of topiramate, wherein the drug is not topiramate.

18. A method of determining an amount of topiramate in a sample comprising
(a) contacting the sample with an antibody raised against the compound of claim 2, thus yielding an antibody-topiramate complex; and
(b) detecting the antibody-topiramate complex.

19. The method of claim 18 further comprising contacting the sample with a ligand competitively binding to the antibody.

20. The method of claim 19 wherein the ligand is the compound of claim 2.

21. A kit for determining the amount of topiramate in a sample, the kit comprising an antibody raised against the compound of claim 2 and ancillary reagents.
